(19) United States
(12) Patent Application Publication
(10) Pub. No.: US 2022/0202828 A1
(43) Pub. Date: Jun. 30, 2022

(54) INSULIN-LIKE GROWTH FACTOR AXIS PROTEINS AS CIRCULATING PULMONARY ARTERIAL HYPERTENSION BIOMARKERS

(71) Applicant: THE JOHNS HOPKINS UNIVERSITY, Baltimore, MD (US)

(72) Inventors: Allen Dale Everett, Glenwood, MD (US); Jun Yang, Baltimore, MD (US)

(21) Appl. No.: 17/607,555

(22) PCT Filed: Apr. 29, 2020
(86) PCT No.: PCT/US2020/030389

§ 371 Date: Oct. 29, 2021

Related U.S. Application Data
(60) Provisional application No. 62/839,991, filed on Apr. 29, 2019.

(51) Int. Cl. A61K 31/5585; G01N 33/68; A61P 9/12

(52) U.S. Cl. CPC A61K 31/5585; G01N 33/6872; G01N 2800/321; A61P 9/12; G01N 33/6893

(57) ABSTRACT

The present invention relates to the field of pulmonary arterial hypertension. More specifically, the present invention provides methods and compositions useful in assessing Insulin-Like Growth Factor (IGF) axis proteins. In one embodiment, a method comprises the steps of (a) detecting an increased level of IGFBP2 relative to a control in a sample obtained from a subject suspected of having PAH; and (b) treating the subject with a PAH therapy.

### CROSS-REFERENCE TO RELATED APPLICATIONS

This application claims the benefit of U.S. Provisional Application No. 62/839,991, filed Apr. 29, 2019, which is incorporated herein by reference in its entirety.

### FIELD OF THE INVENTION

The present invention relates to the field of pulmonary arterial hypertension. More specifically, the present invention provides methods and compositions useful in assessing Insulin-Like Growth Factor (IGF) axis proteins.

### BACKGROUND OF THE INVENTION

Pulmonary arterial hypertension (PAH) is a fatal disease, characterized by a resting mean pulmonary arterial pressure of 25 mm Hg or above. PAH is an extremely heterogeneous disease, classified into seven groups by the Sixth World Symposium on Pulmonary Hypertension in 2019. Although its precise etiology is not well understood, the development of progressive pulmonary vascular resistance and associated right heart failure are common pathologic features. PAH treatment options have greatly expanded in the last half century, improving patients' functional capacity and hemodynamics. However, PAH therapies focus on dynamic pulmonary vasoconstriction as the only mechanism of disease, thus, the five year mortality remains unchanged at approximately 50% for the past 20 years.

Early detection of PAH is difficult, as the disease is typically quite advanced before symptoms emerge. Measurement of the commonly used blood marker N-terminal pro-brain natriuretic peptide (NTproBNP), a marker of cardiac function and stretch not related to PAH etiology, is confounded by other factors such as left heart disease and renal function. Thus, a more specific, precise and causally related biomarker which could improve non-invasive diagnosis and monitoring of disease progression is urgently needed.

### SUMMARY OF THE INVENTION

In one aspect, the present invention provides compositions and methods for measuring one or more IGF axis proteins. In one embodiment, the IGF axis proteins comprise insulin-like growth factor binding protein 2 (IGFBP2). In another embodiment, the IGF axis proteins comprise IGF1, IGF2, IGFBP1, IGFBP3, IGFBP4, IGFBP5, IGFBP6, and IGFBP7. In a specific embodiment, the IGF axis proteins comprise IGF1, IGF2 and IGFBP2. In another embodiment, the IGF axis proteins comprise IGF1, IGF2, IGFBP1, and IGFBP2. In yet another embodiment, the IGF axis proteins comprise IGF1, IGF2, IGFBP2 and IGFBP4. In a further embodiment, the IGF axis proteins comprise IGF1, IGF2, IGFBP1, IGFBP2 and IGFBP4. In another embodiment, the IGF axis proteins comprise IGF1, IGF2, IGFBP2 and IGFBP7. In certain embodiment, the IGF axis proteins comprise IGF1, IGF2, IGFBP2 and one or more of IGFBP1, IGFBP4 and IGFBP7. The measured proteins can be detected as being increased or decreased relative to controls. For example, IGFBP1, IGFBP2, IGFBP4 and IGFBP7 can be detected as being increased relative to controls. IGF1 and IGF2 can be detected as being decreased relative to controls.

In another aspect, the measured IGF axis proteins can be used further to determine certain aspects associated with PAH. For example, the measured proteins can be used to identify a subject as having PAH. In certain embodiments, the proteins can be used to assess PAH severity (e.g., at least IGFBP2), predict survival (e.g., at least IGFBP2 and/or IGFBP4), and assess response to therapy.

In certain embodiments, the IGF axis proteins are measured and one or more tests are performed including, but not limited to, echocardiogram, chest X-ray, electrocardiogram, right heart catheterization and blood tests. Additional tests include computerized tomography (CT) scan, magnetic resonance imaging (MRI), pulmonary function test, polysomnogram, ventilation/perfusion (V/Q) scan and open biopsy.

In yet another aspect, the measured IGF axis proteins can be practically applied to treat PAH in a subject. Treatment can include one or more of endothelial receptor antagonists, prostacyclin pathway agonists, nitric oxide-cyclic guanosine monophosphate (NO-cGMP) enhancers, vasodilators, calcium channel blockers, anticoagulants, and diuretics. In certain embodiments, treatment can include oxygen. In a further embodiment, treatment can include stem cell therapy. In another embodiment, treating can comprise surgery including, but not limited to, atrial septostomy and lung and heart transplants.

In particular embodiments, oral treatment options can include endothelin receptor antagonist (ambresantan, bosentan, and macitenatan), phosphodiesteriase inhibitors (PDE5 inhibitors) (sildenafil and tadalafil), prostacyclin analogs (traprostinil), selective IP receptor agonists (selexipag), and soluble guanylate cyclase (sGC) stimulators (riociguat). In other embodiments, inhaled treatment options include iloprost and treprostinil. Intravenous treatment options include treprestinil and epoprostenol. Subcutaneous treatment options include, but are not limited to, treprostinil. More specific treatment regimens are described further herein.

In one embodiment, the present invention provides a method comprising the step of measuring insulin-like growth factor binding protein 2 (IGFBP2) in a sample obtained from a subject. In particular embodiments, the sample is a serum sample. In another embodiment, the method further comprises measuring IGF1 and/or IGF2. In certain embodiments, the method further comprises measuring one or more of IGFBP1, IGFBP3, IGFBP4, IGFBP5, IGFBP6, and IGFBP7. In another specific embodiment, the method further comprises measuring IGFBP1 and IGFBP4. In yet another specific embodiment, the method further comprises measuring IGF1, IGF2, IGFBP1 and IGFBP4. In the methods of the present invention, the measuring step is performed using an immunoassay. For example, the immunoassay comprises enzyme linked immunosorbent assay (ELISA). In certain embodiments, the subject is suspected of having or has pulmonary arterial hypertension (PAH).

The present invention also provides a method for identifying subject as having PAH comprising the step of measuring IGFBP2 in a sample obtained from the subject, wherein an increased level of IGFBP2 relative to a control identifies the subject as having PAH. In particular embodiments, the sample is a serum sample. In another embodiment, the method further comprises measuring IGF1 and/or IGF2. In certain embodiments, the method further comprises measuring one or more of IGFBP1, IGFBP3, IGFBP4, IGFBP5, IGFBP6, and IGFBP7. In another specific embodiment, the method further comprises measuring IGFBP1 and IGFBP4, wherein an increased level of IGFBP1 and IGFBP4 relative to controls identifies the subject as having PAH. In certain embodiments, the method further comprises measuring IGF1, IGF2, IGFBP1 and IGFBP4 wherein a decreased level of IGF1 and IGF2 and an increased level of IGFBP1 and IGFBP4 relative to controls identifies the subject as having PAH. In the methods of the present invention, the measuring step is performed using an immunoassay. In one embodiment, the immunoassay comprises an ELISA.

In yet another embodiment, the present invention provides a method comprising the steps of (a) detecting an increased level of IGFBP2 relative to a control in a sample obtained from a subject suspected of having PAH; and (b) treating the subject with a PAH therapy. In particular embodiments, the PAH therapy comprising one or more of endothelial receptor antagonists, prostacyclin pathway agonists, nitric oxide-cyclic guanosine monophosphate (NO-cGMP) enhancers, vasodilators, calcium channel blockers, anticoagulants, oxygen and diuretics. In a specific embodiment, the PAH therapy comprises administering prostacyclin or analogs thereof.

### BRIEF DESCRIPTION OF THE FIGURES

FIG. 1. Comparison of receiver operating curves (ROC) for IGF1, IGF2, IGFBP2 and IGFs/IGFBP2 molar ratio as predictors of pulmonary arterial hypertension for 127 subjects from the JHPH cohort and 128 healthy control subjects. The AUC for IGFBP2 is 0.76, 95% confidence interval [CI] 0.698-0.808, p<0.0001; AUC for IGFs/IGFBP2 molar ratio is 0.72, 95% confidence interval [CI] 0.657-0.773, p<0.0001; AUC for IGF1 is 0.52, 95% confidence interval [CI] 0.455-0.583, p=0.61; AUC for IGF2 is 0.50, 95% confidence interval [CI] 0.438-0.566, p=0.96.

FIG. 2. Plot of IGFBP2 level versus REVEAL risk scores. Error bars indicate 25%-75% IQR, black dots indicate median IGFBP2 levels.

FIG. 3A-3B. Kaplan-Meier survival curve for IGFBP2 (FIG. 3A) and IGFs/IGFBP2 molar ratio (FIG. 3B) in JHPH cohort. The curves represent survival analysis of JHPH cohort dichotomized by median serum IGFBP2 (n=125, p=0.0001) or IGFs/IGFBP2 (n=125, p=0.001) levels.

FIG. 4A-4B. Kaplan-Meier survival curve for IGFBP2 (FIG. 4A) and IGFs/IGFBP2 molar ratio (FIG. 4B) in PAHB cohort. The curves represent survival analysis of PAH cohort dichotomized by median serum IGFBP2 (n=219, p<0.0001) or IGFs/IGFBP2 (n=219, p<0.0001) levels.

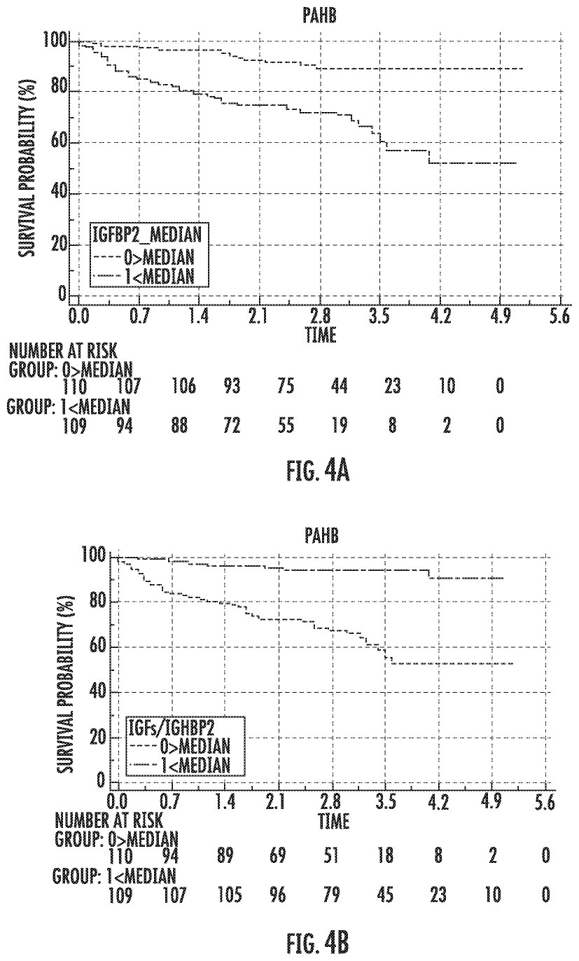

FIG. 5. ROC curve of IGFBP2 in PAH versus Controls. P<0.001.

FIG. 6A-6B. Circulating IGFBP2 (FIG. 6A) and Total IGF/IGFBP2 ratio (FIG. 6B) in pediatric PAH. HR 8.005 for IGFBP2 above median (p=0.003) and HR 0.199 for Total IGF/IGFBP2 ratio above the median (p=0.003). Blue lines above median, red lines below median.

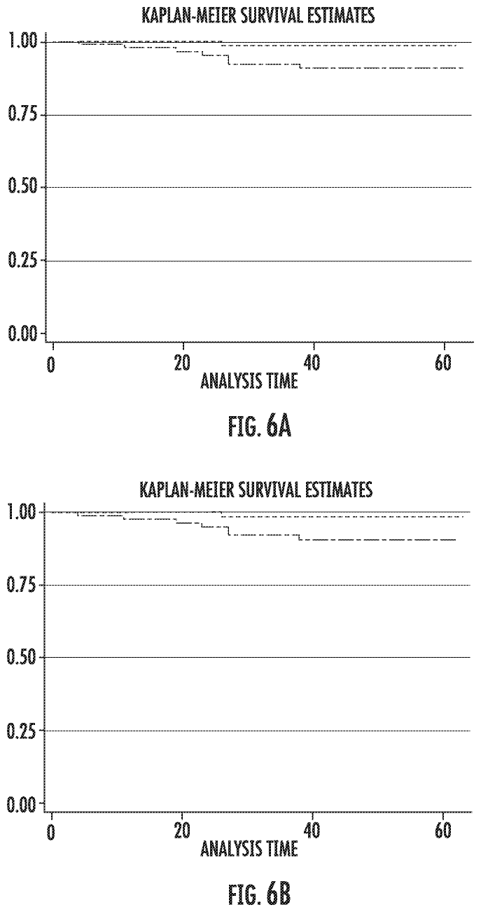

FIG. 7A-7C. Serum concentrations of IGFBP1 (FIG. 7A), IGFBP2 (FIG. 7B) and IGFBP4 (FIG. 7C) in JHPH and control using IGFBP multiplex ELISAs.

FIG. 8. Kaplan-Meier survival analysis of IGFBP4 in the JHPH cohort (N=42). P=0.002.

FIG. 9. Venn diagram of protein identification overlap between IPAH and control cohorts.

FIG. 10. PCA scores plot of overlap of IPAH (Red) and control (Green) protein datasets (File: pca_score2d_0_dpi72).

FIG. 11. Heat map analysis of protein clustering in IPAH and control cohorts (File: heatmap_3_dpi72). Control=0 and IPAH=1.

FIG. 12. All members of the IGF axis were identified by the MS discovery. IGFBP2 had the greatest difference between PH and control, with IGFBP1, 4, and 7 also increased. However IGF 1 and 2 were reduced.

FIG. 13. IGFBP2 ELISA verification in adult PAH (N=36) and controls (N=35). Values are median with IQR.

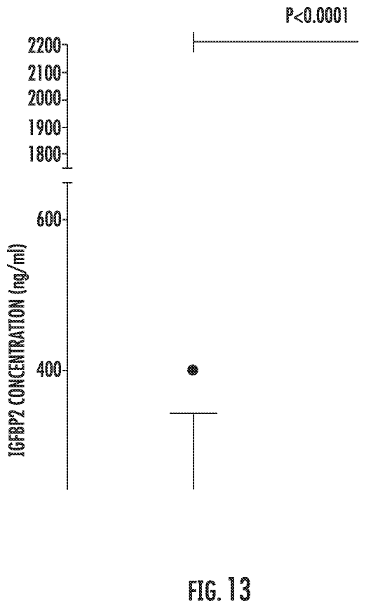

FIG. 14. Box and whisker plot of serum IGF1 and IGF2 levels in control (N=35) and PAH (N=36). Boxes represent the interquartile range (IQR 5-95%) and the horizontal lines are the medians. IGF2, P<0.008.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, wood chucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets. In various embodiments, the subject is mouse or mice. In various embodiments, the subject is human.

A subject can be one who has been previously diagnosed with or identified as suffering from or having a condition, disease, or disorder in need of treatment (e.g., PAH) or one or more complications related to the condition, disease, or disorder, and optionally, have already undergone treatment for the condition, disease, disorder, or the one or more complications related to the condition, disease, or disorder. Alternatively, a subject can also be one who has not been previously diagnosed as having a condition, disease, or disorder or one or more complications related to the condition, disease, or disorder. For example, a subject can be one who exhibits one or more risk factors for a condition, disease, or disorder, or one or more complications related to the condition, disease, or disorder, or a subject who does not exhibit risk factors. A “subject in need” of treatment for a particular condition, disease, or disorder can be a subject suspected of having that condition, disease, or disorder, diagnosed as having that condition, disease, or disorder, already treated or being treated for that condition, disease, or disorder, not treated for that condition, disease, or disorder, or at risk of developing that condition, disease, or disorder.

In some embodiments, the subject is selected from the group consisting of a subject suspected of having a disease, a subject that has a disease, a subject diagnosed with a disease, a subject that has been treated for a disease, a subject that is being treated for a disease, and a subject that is at risk of developing a disease.

In some embodiments, the subject is selected from the group consisting of a subject suspected of having PAH, a subject that has PAH, a subject diagnosed with PAH, a subject that has been treated for PAH, a subject that is being treated for PAH, and a subject that is at risk of developing PAH.

By “at risk of” is intended to mean at increased risk of, compared to a normal subject, or compared to a control group, e.g., a patient population. Thus, a subject carrying a particular marker may have an increased risk for a specific condition, disease or disorder, and be identified as needing further testing. “Increased risk” or “elevated risk” mean any statistically significant increase in the probability, e.g., that the subject has the disorder. The risk is increased by at least 10%, at least 20%, and even at least 50% over the control group with which the comparison is being made.

“Sample” is used herein in its broadest sense. The term “biological sample” as used herein denotes a sample taken or isolated from a biological organism. A sample or biological sample may comprise a bodily fluid including blood, serum, plasma, tears, aqueous and vitreous humor, spinal fluid; a soluble fraction of a cell or tissue preparation, or media in which cells were grown; or membrane isolated or extracted from a cell or tissue; polypeptides, or peptides in solution or bound to a substrate; a cell; a tissue, a tissue print, a fingerprint, skin or hair; fragments and derivatives thereof. Non-limiting examples of samples or biological samples include cheek swab; mucus; whole blood, blood, serum; plasma; urine; saliva, semen; lymph; fecal extract; sputum; other body fluid or biofluid; cell sample; and tissue sample etc. The term also includes a mixture of the above-mentioned samples or biological samples. The term “sample” also includes untreated or pretreated (or pre-processed) biological samples. In some embodiments, a sample or biological sample can comprise one or more cells from the subject. Subject samples or biological samples usually comprise derivatives of blood products, including blood, plasma and serum. In some embodiments, the sample is a biological sample. In some embodiments, the sample is blood. In some embodiments, the sample is plasma. In some embodiments, the sample is blood, plasma, serum, or urine. In certain embodiments, the sample is a serum sample.

The terms “body fluid” or “bodily fluids” are liquids originating from inside the bodies of organisms. Bodily fluids include amniotic fluid, aqueous humour, vitreous humour, bile, blood (e.g., serum), breast milk, cerebrospinal fluid, cerumen (earwax), chyle, chyme, endolymph and perilymph, exudates, feces, female ejaculate, gastric acid, gastric juice, lymph, mucus (e.g., nasal drainage and phlegm), pericardial fluid, peritoneal fluid, pleural fluid, pus, rheum, saliva, sebum (skin oil), serous fluid, semen, sputum, synovial fluid, sweat, tears, urine, vaginal secretion, and vomit. Extracellular bodily fluids include intravascular fluid (blood plasma), interstitial fluids, lymphatic fluid and transcellular fluid. “Biological sample” also includes a mixture of the above-mentioned body fluids. “Biological samples” may be untreated or pretreated (or pre-processed) biological samples.

Sample collection procedures and devices known in the art are suitable for use with various embodiment of the present invention. Examples of sample collection procedures and devices include but are not limited to: phlebotomy tubes (e.g., a vacutainer blood/specimen collection device for collection and/or storage of the blood/specimen), dried blood spots, Microvette CB300 Capillary Collection Device (Sarstedt), HemaXis blood collection devices (microfluidic technology, Hemaxis), Volumetric Absorptive Microsampling (such as CE-IVD Mitra microsampling device for accurate dried blood sampling (Neoteryx), HemaSpot™-HF Blood Collection Device, a tissue sample collection device; standard collection/storage device (e.g., a collection/storage device for collection and/or storage of a sample (e.g., blood, plasma, serum, urine, etc.); a dried blood spot sampling device. In some embodiments, the Volumetric Absorptive Microsampling (VAMS^{lM}) samples can be stored and mailed, and an assay can be performed remotely.

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, -carboxyglutamate, and O-phosphoserine. Amino acid analogs refers to compounds that have the same basic chemical structure as a naturally occurring amino acid, i.e., an carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function s in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “peptide” as used herein refers to any compound containing at least two amino acid residues joined by an amide bond formed from the carboxyl group of one amino acid residue and the amino group of the adjacent amino acid residue. In some embodiments, peptide refers to a polymer of amino acid residues typically ranging in length from 2 to about 30, or to about 40, or to about 50, or to about 60, or to about 70 residues. In certain embodiments the peptide ranges in length from about 2, 3, 4, 5, 7, 9, 10, or 11 residues to about 60, 50, 45, 40, 45, 30, 25, 20, or 15 residues. In certain embodiments the peptide ranges in length from about 8, 9, 10, 11, or 12 residues to about 15, 20 or 25 residues. In some embodiments, the peptide ranges in length from 2 to about 12 residues, or 2 to about 20 residues, or 2 to about 30 residues, or 2 to about 40 residues, or 2 to about 50 residues, or 2 to about 60 residues, or 2 to about 70 residues. In certain embodiments the amino acid residues comprising the peptide are “L-form” amino acid residues, however, it is recognized that in various embodiments, “D” amino acids can be incorporated into the peptide. Peptides also include amino acid polymers in which one or more amino acid residues are an artificial chemical analogue of a corresponding naturally occurring amino acid, as well as to naturally occurring amino acid polymers. In addition, the term applies to amino acids joined by a peptide linkage or by other, “modified linkages” (e.g., where the peptide bond is replaced by an a-ester, a f3-ester, a thioamide, phosphonamide, carbamate, hydroxylate, and the like (see, e.g., Spatola, (1983) Chem. Biochem. Amino Acids and Proteins 7: 267-357), where the amide is replaced with a saturated amine (see, e.g., Skiles et al., U.S. Pat. No. 4,496,542, which is incorporated herein by reference, and Kaltenbronn et al., (1990) pp. 969-970 in Proc. '11th American Peptide Symposium, ESCOM Science Publishers, The Netherlands, and the like)).

A protein refers to any of a class of nitrogenous organic compounds that comprise large molecules composed of one or more long chains of amino acids and are an essential part of all living organisms. A protein may contain various modifications to the amino acid structure such as disulfide bond formation, phosphorylations and glycosylations. A linear chain of amino acid residues may be called a “polypeptide,” A protein contains at least one polypeptide. Short polypeptides, e.g., containing less than 20-30 residues, are sometimes referred to as “peptides.”

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy-chain variable region.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity).

The term “threshold” as used herein refers to the magnitude or intensity that must be exceeded for a certain reaction, phenomenon, result, or condition to occur or be considered relevant. The relevance can depend on context, e.g., it may refer to a positive, reactive or statistically significant relevance.

By “binding assay” is meant a biochemical assay wherein the biomarkers are detected by binding to an agent, such as an antibody, through which the detection process is carried out. The detection process may involve fluorescent or radioactive labels, and the like. The assay may involve immobilization of the biomarker, or may take place in solution.

“Immunoassay” is an assay that uses an antibody to specifically bind an antigen (e.g., a marker). The immunoassay is characterized by the use of specific binding properties of a particular antibody to isolate, target, and/or quantify the antigen. Non-limiting examples of immunoassays include ELISA (enzyme-linked immunosorbent assay), immunoprecipitation, SISCAPA (stable isotope standards and capture by anti-peptide antibodies), Western blot, etc.

“Diagnostic” means identifying the presence or nature of a pathologic condition, disease, or disorder and includes identifying patients who are at risk of developing a specific condition, disease or disorder. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, a disease, or a disorder, it suffices if the method provides a positive indication that aids in diagnosis.

The term “statistically significant” or “significantly” refers to statistical evidence that there is a difference. It is defined as the probability of making a decision to reject the null hypothesis when the null hypothesis is actually true. The decision is often made using the p-value.

The terms “detection”, “detecting” and the like, may be used in the context of detecting biomarkers, detecting peptides, detecting proteins, or of detecting a condition, detecting a disease or a disorder (e.g., when positive assay results are obtained). In the latter context, “detecting” and “diagnosing” are considered synonymous.

The terms “marker” or “biomarker” are used interchangeably herein, and in the context of the present invention refer to a protein or peptide (for example, protein or peptide associated with PAH or PAH as described herein) is differentially present in a sample taken from patients having a specific disease or disorder as compared to a control value, the control value consisting of, for example average or mean values in comparable samples taken from control subjects (e.g., a person with a negative diagnosis, normal or healthy subject). Biomarkers may be determined as specific peptides or proteins which may be detected by, for example, antibodies or mass spectroscopy. In some applications, for example, a mass spectroscopy or other profile of multiple antibodies may be used to determine multiple biomarkers, and differences between individual biomarkers and/or the partial or complete profile may be used for diagnosis. In some embodiments, the biomarkers may be detected by antibodies, mass spectrometry, or combinations thereof.

A “test amount” of a marker refers to an amount of a marker present in a sample being tested. A test amount can be either in absolute amount (e.g., g/mi) or a relative amount (e.g., relative intensity of signals).

A “diagnostic amount” of a marker refers to an amount of a marker in a subject's sample that is consistent with a diagnosis of a particular disease or disorder. A diagnostic amount can be either in absolute amount (e.g., μg/ml) or a relative amount (e.g., relative intensity of signals).

A “control amount” of a marker can be any amount or a range of amount which is to be compared against a test amount of a marker. For example, a control amount of a marker can be the amount of a marker in a person who does not suffer from the disease or disorder sought to be diagnosed, A control amount can be either in absolute amount (e.g., μg/ml) or a relative amount (e.g., relative intensity of signals).

The term “differentially present” or “change in level” refers to differences in the quantity and/or the frequency of a marker present in a sample taken from patients having a specific disease or disorder as compared to a control subject. For example, a marker can be present at an elevated level or at a decreased level in samples of patients with the disease or disorder compared to a control value (e.g., determined from samples of control subjects). Alternatively, a marker can be detected at a higher frequency or at a lower frequency in samples of patients compared to samples of control subjects. A marker can be differentially present in terms of quantity, frequency or both as well as a ratio of differences between two or more specific modified amino acid residues and/or the protein itself. In one embodiment, an increase in the ratio of modified to unmodified proteins and peptides described herein is diagnostic of any one or more of the diseases described herein. In another embodiment, and as described further herein, a ratio of total IGF (IGF1+IGF2) can be compared to one or more IGF axis proteins including, for example, IGFBP2.

A marker, compound, composition or substance is differentially present in a sample if the amount of the marker, compound, composition or substance in the sample is statistically significantly different from the amount of the marker, compound, composition or substance in another sample, or from a control value. For example, a compound is differentially present if it is present at least about 120%, at least about 130%, at least about 150%, at least about 180%, at least about 200%, at least about 300%, at least about 500%, at least about 700%, at least about 900%, or at least about 1000% greater or less than it is present in the other sample (e.g., control), or if it is detectable in one sample and not detectable in the other.

Alternatively, or additionally, a marker, compound, composition or substance is differentially present between samples if the frequency of detecting the marker, etc. in samples of patients suffering from a particular disease or disorder, is statistically significantly higher or lower than in the control samples or control values obtained from healthy individuals. For example, a biomarker is differentially present between the two sets of samples if it is detected at least about 10%, at least about 20%, at least about 30%, at least about 40%, at least about 50%, at least about 60%, at least about 70%, at least about 80%, at least about 90%, or at least about 100% more frequently or less frequently observed in one set of samples than the other set of samples. These exemplary values notwithstanding, it is expected that a skilled practitioner can determine cut-off points, etc., that represent a statistically significant difference to determine whether the marker is differentially present.

The term “one or more of” refers to combinations of various IGF axis protein biomarkers. The term encompasses 1, 2, 3, 4, 5, 6, 7, 8, 9, 10, 11, 12, 13, 15, 16, 17, 18, 19, 20, 21, 22, 23, 24, 25, 26, 27, 28, 29, 30, 31, 32, 33, 34, 35, 36, 37, 38, 39, 40 . . . N, where “N” is the total number of biomarker proteins in the particular embodiment. The term also encompasses, and is interchangeably used with, at least 1, at least 2, at least 3, at least 4, at least 5, at least 6, at least 7, at least 8, at least 9, at least 10, at least 11, at least 12, at least 13, at least 15, 16, 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40 . . . N. It is understood that the recitation of biomarkers herein includes the phrase “one or more of” the biomarkers and, in particular, includes the “at least 1, at least 2, at least 3” and so forth language in each recited embodiment of a biomarker panel.

“Detectable moiety” or a “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavidin, digoxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, flow cytometry, or direct analysis by mass spectrometry of intact protein or peptides. In some embodiments, the detectable moiety is a stable isotope. In some embodiments, the stable isotope is selected from the group consisting of ¹⁵N, ¹³C, ¹⁸O and ²H.

In various embodiments the invention provides a method to identify protein biomarkers and patterns that are indicative of a disease. In various embodiments the invention provides a method to identify protein biomarkers and patterns that are indicative a disease is or may be present. In some embodiments these methods may provide objective rationale for further testing. In various embodiments the invention provides a method for the identification of a plurality of proteins from a sample, wherein each protein is correlated to one or more peptides, wherein each peptide is correlated to one or more transitions, wherein each transition comprises a Q1 mass value. In various embodiments the invention provides a method for the identification of a plurality of proteins from a sample, wherein each protein is correlated to one or more peptides, wherein each peptide is correlated to one or more transitions, wherein each transition comprises a Q1 mass value and a Q3 mass value. In various embodiments the invention provides a method for the identification of a plurality of proteins from a sample, wherein each protein is correlated to one or more peptides, wherein each peptide is correlated to one or more transitions, wherein each transition comprises a Q1/Q3 mass value pair.

As used herein, SRM stands for selected reaction monitoring. As used herein, MRM stands for multiple reaction monitoring. As used herein, PRM stands for parallel reaction monitoring. As used herein, SWATH stands for sequential window acquisition of all theoretical fragment ion spectra. As used herein, DIA stands for data-independent acquisition. As used herein, MS stands for mass spectrometry. As used herein, SIL stands for stable isotope-labeled.

As used herein, “MS data” can be raw MS data obtained from a mass spectrometer and/or processed MS data in which peptides and their fragments (e.g., transitions and MS peaks) are already identified, analyzed and/or quantified. MS data can be Selective Reaction Monitoring (SRM) data, Multiple Reaction Monitoring (MRM) data, parallel reaction monitoring (PRM) data, Shotgun CID MS data, Original DIA MS Data, MSE MS data, p2CID MS Data, PAcIFIC MS Data, AIF MS Data, XDLA MS Data, SWATH MS data, or FT-ARM MS Data, or their combinations.

In some embodiments of the present invention, based on SRM and/or MS, and/or PRM MS, allows for the detection and accurate quantification of specific peptides in complex mixtures.

Selected Reaction Monitoring or Multiple Reaction Monitoring (SRM/MRM) mass spectrometry is a technology with the potential for reliable and comprehensive quantification of substances of low abundance in complex samples. SRM is performed on triple quadrupole-like instruments, in which increased selectivity is obtained through collision-induced dissociation. It is a non-scanning mass spectrometry technique, where two mass analyzers (Q1 and Q3) are used as static mass filters, to monitor a particular fragment of a selected precursor. On triple quadrapole instruments, various ionization methods can be used including without limitation electrospray ionization, chemical ionization, electron ionization, atmospheric pressure chemical ionization, and matrix-assisted laser desorption ionization. Both the first mass analyzer and the collision cell are continuously exposed to ions from the source in a time dependent manner. Once the ions move into the third mass analyzer time dependence becomes a factor. On triple quadrupole instruments, the first quadrapole mass filter, Q1 is the primary m/z selector after the sample leaves the ionization source. Any ions with mass-to-charge ratios other than the one selected for will not be allowed to infiltrate Q1. The collision cell, denoted as “q2”, located between the first quadrapole mass filter Q1 and second quadrapole mass filter Q3, is where fragmentation of the sample occurs in the presence of an inert gas like argon, helium, or nitrogen. Upon exiting the collision cell, the fragmented ions then travel onto the second quadrapole mass filter Q3, where m/z selection can occur again. The specific pair of mass-over-charge (m/z) values associated to the precursor and fragment ions selected is referred to as a “transition”. The detector acts as a counting device for the ions matching the selected transition thereby returning an intensity distribution over time. MRM is when multiple SRM transitions are measured within the same experiment on the chromatographic time scale by rapidly switching between the different precursor/fragment pairs. Typically, the triple quadrupole instrument cycles through a series of transitions and records the signal of each transition as a function of the elution time. The method allows for additional selectivity by monitoring the chromatographic co-elution of multiple transitions for a given analyte.

In addition to MRM, the choice of peptides can also be quantified through Parallel-Reaction Monitoring (PRM), Parallel reaction monitoring (PRM) is the application of SRM with parallel detection of all transitions in a single analysis using a high resolution mass spectrometer. PRM provides high selectivity, high sensitivity and high-throughput to quantify selected peptide (Q1), hence quantify proteins. Again, multiple peptides can be specifically selected for each protein. PRM methodology uses the quadrupole of a mass spectrometer to isolate a target precursor ion, fragments the targeted precursor ion in the collision cell, and then detects the resulting product ions in the Orbitrap mass analyzer. Quantification is carried out after data acquisition by extracting one or more fragment ions with 5-10 ppm mass windows. PRM uses a quadrupole time-of-flight (QTOF) or hybrid quadrupole-orbitrap (QOrbitrap) mass spectrometer to carry out the peptides/proteins quantitation. Examples of QTOF include but are not limited to: TripleTOF® 6600 or 5600 System (Sciex); X500R QTOF System (Sciex); 6500 Series Accurate-Mass Quadrupole Time-of-Flight (Q-TOF) (Agilent); or Xevo G2-XS QTof Quadrupole Time-of-Flight Mass Spectrometry (Waters). Examples of QObitrap include but are not limited to: Q Exactive™ Hybrid Quadrupole-Orbitrap Mass Spectrometer (the Thermo Scientific); or Orbitrap Fusion™ Tribrid™ (the Thermo Scientific).

Non-limiting advantages of PRM include elimination of most interferences, provides more accuracy and attomole-level limits of detection and quantification, enables the confident confirmation of the peptide identity with spectral library matching, reduces assay development time since no target transitions need to be preselected, ensures UHPLC-compatible data acquisition speeds with spectrum multiplexing and advanced signal processing.

SWATH MS is a data independent acquisition (DIA) method which aims to complement traditional mass spectrometry-based proteomics techniques such as shotgun and SRM methods. In essence, it allows a complete and permanent recording of all fragment ions of the detectable peptide precursors present in a biological sample. It thus combines the advantages of shotgun (high throughput) with those of SRM (high reproducibility and consistency).

In some embodiments, the developed methods herein can be applied to the quantification of polypeptides(s) or protein(s) in biological sample(s). Any kind of biological samples comprising polypeptides or proteins can be the starting point and be analyzed by the methods herein. Indeed, any protein/peptide containing sample can be used for and analyzed by the methods produced here (e.g., tissues, cells). The methods herein can also be used with peptide mixtures obtained by digestion. Digestion of a polypeptide or protein includes any-kind of cleavage strategies such as enzymatic, chemical, physical or combinations thereof.

In some embodiments, the analysis and/or comparison is performed on protein samples of wild-type or physiological/healthy origin against protein samples of mutant or pathological origin.

As used herein, the terms “treat”, “treatment”, “treating”, or “amelioration” when used in reference to a disease, disorder or medical condition, refer to both therapeutic treatment and prophylactic or preventative measures, wherein the object is to reverse, alleviate, ameliorate, inhibit, lessen, slow down or stop the progression or severity of a symptom, a condition, a disease, or a disorder. The term “treating” includes reducing or alleviating at least one adverse effect or symptom of a condition, a disease, or a disorder. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition, disease, or disorder even if the treatment is ultimately unsuccessful. Those in need of treatment include those already with the condition, disease, or disorder as well as those prone to have the condition, disease, or disorder or those in whom the condition, disease, or disorder is to be prevented.

Non-limiting examples of treatments or therapeutic treatments include pharmacological or biological therapies and/or interventional surgical treatments.

The term “preventative treatment” means maintaining or improving a healthy state or non-diseased state of a healthy subject or subject that does not have a disease. The term “preventative treatment” or “health surveillance” also means to prevent or to slow the appearance of symptoms associated with a condition, disease, or disorder. The term “preventative treatment” also means to prevent or slow a subject from obtaining a condition, disease, or disorder.

As used herein, the term “administering,” refers to the placement an agent or a treatment as disclosed herein into a subject by a method or route which results in at least partial localization of the agent or treatment at a desired site. “Route of administration” may refer to any administration pathway known in the art, including but not limited to aerosol, nasal, via inhalation, oral, anal, intra-anal, peri-anal, transmucosal, transdermal, parenteral, enteral, topical or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrastemai, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions can be in the form of aerosol, lotion, cream, gel, ointment, suspensions, solutions or emulsions. In accordance with the present invention, “administering” can be self-administering. For example, it is considered as “administering” that a subject consumes a composition as disclosed herein.

In one aspect, the present invention provides compositions and methods for measuring one or more IGF axis proteins. In one embodiment, the IGF axis proteins comprise IGFBP2. In another embodiment, the IGF axis proteins comprise IGF1, IGF2, IGFBP1, IGFBP3, IGFBP4, IGFBP5, IGFBP6, and IGFBP7. In a specific embodiment, the IGF axis proteins comprise IGF1, IGF2 and IGFBP2. In another embodiment, the IGF axis proteins comprise IGF1, IGF2, IGFBP1, and IGFBP2. In yet another embodiment, the IGF axis proteins comprise IGF1, IGF2, IGFBP2 and IGFBP4. In a further embodiment, the IGF axis proteins comprise IGF1, IGF2, IGFBP1, IGFBP2 and IGFBP4. In another embodiment, the IGF axis proteins comprise IGF1, IGF2, IGFBP2 and IGFBP7. In certain embodiment, the IGF axis proteins comprise IGF1, IGF2, IGFBP2 and one or more of IGFBP1, IGFBP4 and IGFBP7. The measured proteins can be detected as being increased or decreased relative to controls. For example, IGFBP1, IGFBP2, IGFBP4 and IGFBP7 can be detected as being increased relative to controls. IGF1 and IGF2 can be detected as being decreased relative to controls.

In another aspect, the measured IGF axis proteins can be used further to determine certain aspects associated with PAH. For example, the measured proteins can be used to identify a subject as having PAH. In certain embodiments, the proteins can be used to assess PAH severity (e.g., IGFBP2), predict survival (e.g., IGFBP2 and/or IGFBP4), and predict response to therapy.

In certain embodiments, the IGF axis proteins are measured and one or more tests are performed including, but not limited to, echocardiogram, chest X-ray, electrocardiogram, right heart catheterization and blood tests. Additional tests include computerized tomography (CT) scan, magnetic resonance imaging (MRI), pulmonary function test, polysomnogram, ventilation/perfusion (V/Q) scan and open biopsy.

In yet another aspect, the measured IGF axis proteins can be practically applied to treat PAH in a subject. Treatment can include endothelial receptor antagonists, prostacyclin pathway agonists, nitric oxide-cyclic guanosine monophosphate (NO-cGMP) enhancers, vasodilators, calcium channel blockers, anticoagulants, and diuretics. In certain embodiments, endothelin receptor antagonists include, but are not limited to, ambrisentan (LETAIRIS®) (Gilead Sciences, Inc.), macitentan (OPSUMIT®) (Actelion Pharmaceuticals, Inc.), bosentan (TRACLEER®) (Actelion Pharmaceuticals, Inc.). In other embodiments, prostacyclin pathway agonists include, but are not limited to prostacyclin (epoprostenol), synthetic analogs of prostacyclin (intravenous treprostinil, subcutaneous treprostinil, inhaled treprostinil, and inhaled iloprost), and selexipag (UPTRAVI®) (Actelion Pharmaceuticals, Inc.).

NO-cGMP enhancers include, but are not limited to PDE5 inhibitors (sildenafil (REVATIO®/VIAGRA®), tadalafil (ADCIRCA®/CIALIS®), and vardenafil (LEVITRA®/STAXYN®)) and soluble gyanylate cyclase stimulators (riociguat (ADEMPAS®) (Bayer). Vasodilators include, but are not limited to, epoprostenol (FLOLAN®/VELETRI®)), iloprost (VENTAVIS®), and treprostinil (TYVASO®/REMODULIN®, ORENITRAM®). Calcium channel blockers include, but are not limited to amlodipine (NORVASC), dilitiazem (CARDIZEM®/TIAZAC®), and nifedipine (PROCARDIA®). In particular embodiments, anticoagulants include, but are not limited to warfarin (COUMADIN®/JANTOVEN®). In particular embodiments, treatment comprises digoxin (LANOXIN®).

In certain embodiments, treatment can comprise combination therapy including, but not limited to, tadalafil plus ambrisentan; sildenafil plus bosentan; bosentan added to either epoprostenol or treprostinil; treprostinil added to either bosentan or sildenafil; oral treprostinil added to an endothelin receptor antagonist and/or a phostphodiesterase-5 inhibitor; sildenafil added to epoprostenol; sildenafil added to iloprost; and bosenan plus iloprost; riociguat added to sildenafil.

A physician can determine whether/how the treatments can be administered. For example, in certain embodiments, PAH can be treat using a continuously infused prostacyclins or analogs thereof. Continuous IV or SQ infusions can include FLOLAN®, REMODULIN® and VELETRI®.

In a specific embodiment, treatment comprises stem cell therapy. In further embodiments, treatment can comprise surgery including atrial septostomy and lung and heart transplants.

In specific embodiments, the IGF axis biomarker proteins of the present invention can be detected and/or measured by immunoassay. Immunoassay requires biospecific capture reagents/binding agent, such as antibodies, to capture the biomarkers. Many antibodies are available commercially. Antibodies also can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well-known in the art.

The present invention contemplates traditional immunoassays including, for example, sandwich immunoassays including ELISA or fluorescence-based immunoassays, immunoblots, Western Blots (WB), as well as other enzyme immunoassays. Nephelometry is an assay performed in liquid phase, in which antibodies are in solution. Binding of the antigen to the antibody results in changes in absorbance, which is measured. In a SELDI-based immunoassay, a biospecific capture reagent for the biomarker is attached to the surface of an MS probe, such as a pre-activated protein chip array. The biomarker is then specifically captured on the biochip through this reagent, and the captured biomarker is detected by mass spectrometry.

In certain embodiments, the expression levels of the IGF axis protein biomarkers employed herein are quantified by immunoassay, such as enzyme-linked immunoassay (ELISA) technology. In specific embodiments, the levels of expression of the biomarkers are determined by contacting the biological sample with antibodies, or antigen binding fragments thereof, that selectively bind to the biomarker; and detecting binding of the antibodies, or antigen binding fragments thereof, to the biomarkers. In certain embodiments, the binding agents employed in the disclosed methods and compositions are labeled with a detectable moiety. In other embodiments, a binding agent and a detection agent are used, in which the detection agent is labeled with a detectable moiety.

For example, the level of a biomarker in a sample can be assayed by contacting the biological sample with an antibody, or antigen binding fragment thereof, that selectively binds to the target IGF axis protein (referred to as a capture molecule or antibody or a binding agent), and detecting the binding of the antibody, or antigen-binding fragment thereof, to the IGF axis protein. The detection can be performed using a second antibody to bind to the capture antibody complexed with its target biomarker. A target biomarker can be an entire protein, or a variant or modified form thereof. Kits for the detection of IGF axis proteins as described herein can include pre-coated strip/plates, biotinylated secondary antibody, standards, controls, buffers, streptavidin-horse radish peroxidise (HRP), tetramethyl benzidine (TMB), stop reagents, and detailed instructions for carrying out the tests including performing standards.

The present disclosure also provides methods for detecting IGF axis protein in a sample obtained from a subject, wherein the levels of expression of the IGF axis proteins in a biological sample are determined simultaneously. For example, in one embodiment, methods are provided that comprise: (a) contacting a biological sample obtained from the subject with a plurality of binding agents that each selectively bind to one or more IGF axis biomarker proteins for a period of time sufficient to form binding agent-biomarker complexes; and (b) detecting binding of the binding agents to the one or more IGF axis biomarker proteins. In further embodiments, detection thereby determines the levels of expression of the biomarkers in the biological sample; and the method can further comprise (c) comparing the levels of expression of the one or more IGF axis biomarker proteins in the biological sample with predetermined threshold values, wherein levels of expression of at least one of the IGF axis biomarker proteins above or below the predetermined threshold values indicates, for example, the subject has PAH, the severity of PAH, and/or is/will be responsive to PAH therapy. Examples of binding agents that can be effectively employed in such methods include, but are not limited to, antibodies or antigen-binding fragments thereof, aptamers, lectins and the like.

Although antibodies are useful because of their extensive characterization, any other suitable agent (e.g., a peptide, an aptamer, or a small organic molecule) that specifically binds a biomarker of the present invention is optionally used in place of the antibody in the above described immunoassays. For example, an aptamer that specifically binds a biomarker and/or one or more of its breakdown products might be used. Aptamers are nucleic acid-based molecules that bind specific ligands. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020.

In specific embodiments, the assay performed on the biological sample can comprise contacting the biological sample with one or more capture agents (e.g., antibodies, peptides, aptamer, etc., combinations thereof) to form a biomarker:capture agent complex. The complexes can then be detected and/or quantified. A subject can then be identified as having PAH based on a comparison of the detected/quantified/measured levels of biomarkers to one or more reference controls as described herein.

In one method, a first, or capture, binding agent, such as an antibody that specifically binds the IGF axis protein biomarker of interest, is immobilized on a suitable solid phase substrate or carrier. The test biological sample is then contacted with the capture antibody and incubated for a desired period of time. After washing to remove unbound material, a second, detection, antibody that binds to a different, non-overlapping, epitope on the biomarker (or to the bound capture antibody) is then used to detect binding of the polypeptide biomarker to the capture antibody. The detection antibody is preferably conjugated, either directly or indirectly, to a detectable moiety. Examples of detectable moieties that can be employed in such methods include, but are not limited to, cheminescent and luminescent agents; fluorophores such as fluorescein, rhodamine and eosin; radioisotopes; colorimetric agents; and enzyme-substrate labels, such as biotin.

In another embodiment, the assay is a competitive binding assay, wherein labeled IGF axis protein biomarker is used in place of the labeled detection antibody, and the labeled biomarker and any unlabeled biomarker present in the test sample compete for binding to the capture antibody. The amount of biomarker bound to the capture antibody can be determined based on the proportion of labeled biomarker detected.

Solid phase substrates, or carriers, that can be effectively employed in such assays are well known to those of skill in the art and include, for example, 96 well microtiter plates, glass, paper, and microporous membranes constructed, for example, of nitrocellulose, nylon, polyvinylidene difluoride, polyester, cellulose acetate, mixed cellulose esters and polycarbonate. Suitable microporous membranes include, for example, those described in US Patent Application Publication no. US 2010/0093557 A1. Methods for the automation of immunoassays are well known in the art and include, for example, those described in U.S. Pat. Nos. 5,885,530, 4,981,785, 6,159,750 and 5,358,691.

The presence of several different IGF axis protein biomarkers in a test sample can be detected simultaneously using a multiplex assay, such as a multiplex ELISA. Multiplex assays offer the advantages of high throughput, a small volume of sample being required, and the ability to detect different proteins across a board dynamic range of concentrations.

In certain embodiments, such methods employ an array, wherein multiple binding agents (for example capture antibodies) specific for multiple biomarkers are immobilized on a substrate, such as a membrane, with each capture agent being positioned at a specific, pre-determined, location on the substrate. Methods for performing assays employing such arrays include those described, for example, in US Patent Application Publication nos. US2010/0093557A1 and US2010/0190656A1, the disclosures of which are hereby specifically incorporated by reference.

Multiplex arrays in several different formats based on the utilization of, for example, flow cytometry, chemiluminescence or electron-chemiluminesence technology, can be used. Flow cytometric multiplex arrays, also known as bead-based multiplex arrays, include the Cytometric Bead Array (CBA) system from BD Biosciences (Bedford, Mass.) and multi-analyte profiling (xMAP®) technology from Luminex Corp. (Austin, Tex.), both of which employ bead sets which are distinguishable by flow cytometry. Each bead set is coated with a specific capture antibody. Fluorescence or streptavidin-labeled detection antibodies bind to specific capture antibody-biomarker complexes formed on the bead set. Multiple biomarkers can be recognized and measured by differences in the bead sets, with chromogenic or fluorogenic emissions being detected using flow cytometric analysis.

In an alternative format, a multiplex ELISA from Quansys Biosciences (Logan, Utah) coats multiple specific capture antibodies at multiple spots (one antibody at one spot) in the same well on a 96-well microtiter plate. Chemiluminescence technology is then used to detect multiple biomarkers at the corresponding spots on the plate.

In several embodiments, the IGF axis protein biomarkers of the present invention may be detected by means of an electrochemicaluminescent assay developed by Meso Scale Discovery (Gaithersrburg, Md.). Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at ˜620 nm, eliminating problems with color quenching. See U.S. Pat. No. 7,497,997; 7,491,540; 7,288,410; 7,036,946; 7,052,861; 6,977,722; 6,919,173; 6,673,533; 6,413,783; 6,362,011; 6,319,670; 6,207,369; 6,140,045; 6,090,545; and 5,866,434. See also U.S. Patent Applications Publication No. 2009/0170121; No. 2009/006339; No. 2009/0065357; No. 2006/0172340; No. 2006/0019319; No. 2005/0142033; No. 2005/0052646; No. 2004/0022677; No. 2003/0124572; No. 2003/0113713; No. 2003/0003460; No. 2002/0137234; No. 2002/0086335; and No. 2001/0021534.

The IGF axis proteins of the present invention can be detected by other suitable methods. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Furthermore, a sample may also be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, Calif.), Invitrogen Corp. (Carlsbad, Calif.), Affymetrix, Inc. (Fremong, Calif.), Zyomyx (Hayward, Calif.), R&D Systems, Inc. (Minneapolis, Minn.), Biacore (Uppsala, Sweden) and Procognia (Berkshire, UK). Examples of such protein biochips are described in the following patents or published patent applications: U.S. Pat. Nos. 6,537,749; 6,329,209; 6,225,047; 5,242,828; PCT International Publication No. WO 00/56934; and PCT International Publication No. WO 03/048768.

In a particular embodiment, the present invention comprises a microarray chip. More specifically, the chip comprises a small wafer that carries a collection of binding agents bound to its surface in an orderly pattern, each binding agent occupying a specific position on the chip. The set of binding agents specifically bind to each of the one or more one or more of the biomarkers described herein. In particular embodiments, a few micro-liters of blood serum or plasma are dropped on the chip array. IGF axis protein biomarkers present in the tested specimen bind to the binding agents specifically recognized by them. Subtype and amount of bound mark is detected and quantified using, for example, a fluorescently-labeled secondary, subtype-specific antibody. In particular embodiments, an optical reader is used for bound biomarker detection and quantification. Thus, a system can comprise a chip array and an optical reader. In other embodiments, a chip is provided.

In another aspect, the present invention provides kits for detecting one or more IGF axis proteins. The exact nature of the components configured in the inventive kit depends on its intended purpose. In one embodiment, the kit is configured particularly for human subjects.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

In various embodiments, the present invention provides a kit comprising: (a) one or more internal standards suitable for measurement of one or more IGF axis proteins including any one or more of mass spectrometry, antibody method, antibodies, nucleic acid aptamer method, nucleic acid aptamers, immunoassay, ELISA, immunoprecipitation, SISCAPA, Western blot, or combinations thereof; and (b) reagents and instructions for sample processing, preparation and IGF axis protein measurement/detection. The kit can further comprise (c) instructions for using the kit to measure IGF axis proteins in a sample obtained from the subject.

In particular embodiments, the kit comprises reagents necessary for processing of samples and performance of an immunoassay. In a specific embodiment, the immunoassay is an ELISA. Thus, in certain embodiments, the kit comprises a substrate for performing the assay (e.g., a 96-well polystyrene plate). The substrate can be coated with antibodies specific for an IGF axis protein. In a further embodiment, the kit can comprise a detection antibody including, for example, a polyclonal antibody specific for an IGF axis protein conjugated to a detectable moiety or label (e.g., horseradish peroxidase). The kit can also comprise a standard, e.g., a human IGFBP2 standard. The kit can also comprise one or more of a buffer diluent, calibrator diluent, wash buffer concentrate, color reagent, stop solution and plate sealers (e.g., adhesive strip).

In particular embodiments, the kit may comprise a solid support, such as a chip, microtiter plate (e.g., a 96-well plate), bead, or resin having IGF axis protein biomarker capture reagents attached thereon. The kit may further comprise a means for detecting the IGF axis protein biomarkers, such as antibodies, and a secondary antibody-signal complex such as horseradish peroxidase (HRP)-conjugated goat anti-rabbit IgG antibody and tetramethyl benzidine (TMB) as a substrate for HRP.

The kit may be provided as an immuno-chromatography strip comprising a membrane on which the antibodies are immobilized, and a means for detecting, e.g., gold particle bound antibodies, where the membrane, includes NC membrane and PVDF membrane. The kit may comprise a plastic plate on which a sample application pad, gold particle bound antibodies temporally immobilized on a glass fiber filter, a nitrocellulose membrane on which antibody bands and a secondary antibody band are immobilized and an absorbent pad are positioned in a serial manner, so as to keep continuous capillary flow of the sample.

In certain embodiments, a subject can be diagnosed by adding a biological sample (e.g., blood) from the patient to the kit and detecting the relevant IGF axis protein biomarkers conjugated with antibodies, specifically, by a method which comprises the steps of: (i) collecting blood from the patient; (ii) adding blood from patient to a diagnostic kit; and, (iii) detecting the IGF axis protein biomarkers conjugated with antibodies. In other kit and diagnostic embodiments, blood will not be collected from the patient (i.e., it is already collected). Blood samples can be collected from subject of varying ages. Moreover, in other embodiments, the sample may comprise a serum, plasma sweat, tissue, urine or a clinical sample.

The kit can also comprise a washing solution or instructions for making a washing solution, in which the combination of the capture reagents and the washing solution allows capture of the IGF axis protein biomarkers on the solid support for subsequent detection by, e.g., antibodies or mass spectrometry. In a further embodiment, a kit can comprise instructions for suitable operational parameters in the form of a label or separate insert. For example, the instructions may inform a consumer about how to collect the sample, etc. In yet another embodiment, the kit can comprise one or more containers with IGF axis protein biomarker samples, to be used as standard(s) for calibration or normalization. Detection of the markers described herein may be accomplished using a lateral flow assay.

In particular embodiments, the IGF axis protein biomarker proteins of the present invention can be captured and concentrated using nano particles. In a specific embodiment, the proteins can be captured and concentrated using Nanotrap® technology (Ceres Nanosciences, Inc. (Manassas, Va.)). Briefly, the Nanotrap platform reduces pre-analytical variability by enabling biomarker enrichment, removal of high-abundance analytes, and by preventing degradation to highly labile analytes in an innovative, one-step collection workflow. Multiple analytes sequestered from a single sample can be concentrated and eluted into small volumes to effectively amplify, up to 100-fold or greater depending on the starting sample volume (Shafagati, 2014; Shafagati, 2013; Longo, et al., 2009), resulting in substantial improvements to downstream analytical sensitivity.

In certain embodiments, the kit comprises reagents and components necessary for performing an electrochemiluminescent ELISA.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1: Insulin-Like Growth Factor Binding Protein-2: A New Circulating Indicator of Pulmonary Arterial Hypertension and Survival

Insulin-like growth factor binding protein 2 (IGFBP2), an important regulator of cardiac and vascular function, is also implicated in pulmonary diseases. The present inventors hypothesized that serum IGFBP2 levels could discriminate pulmonary artery hypertension (PAH) from healthy controls and possibly predict disease severity and survival in PAH. The objective of this study was to determine the diagnostic and prognostic value of serum IGFBP2 in PAH.

Using enzyme-linked immunosorbent assays, the present inventors evaluated serum IGFBP2 levels and total IGF1/2 levels; in 2 PAH cohorts (Johns Hopkins Pulmonar Hypertension, JHPH N=127 and NHLBI PAHBiobank, PAHB N=225) and a healthy control cohort (N=128). Other clinical measures were used to assess the value of IGFBP2 as a PAH severity and survival biomarker.

Serum IGFBP2 levels were significantly elevated (p<0.0001) compared to controls and discriminated PAH from controls with an AUC of 0.76 (p<0.0001). IGFBP2 was significantly associated with a decreased 6MWD in both cohorts and in the PAHB after adjustment for age and gender (coefficient −51.669, p=0.016). IGFBP2 was significantly associated with higher REVEAL risk scores (p<0.0001) in the PAHB cohort. Cox multivariable analysis demonstrated that higher serum IGFBP2 was an independent predictor of survival in both JHPH (hazard ratio [HR] 5.29 (95% CI, 1.61-17.40; p=0.006)) and PAHB cohorts ([HR] 4.12 (95% CI, 1.64-10.37; p=0.003)).

Elevated circulating IGFBP2 levels were significantly associated with PAH diagnosis and prognosis. Considering the significance of IGF to cardiopulmonary function, and without being limited to any particular theory, IGFBP2 may contribute to PAH progression by limiting IGFs availability or through IGF-independent negative regulation of cardiac function and pulmonary vascular tone.

### INTRODUCTION

The Insulin-Like Growth Factor (IGF) axis consists of two hormones (IGF1 and 2), two types of receptors, and 6 binding proteins (IGFBP1-6) with high binding affinity to IGFs (14-16). The IGF system is an evolutionarily conserved system that, together with insulin signaling proteins, coordinates the organisms' metabolic activity in response to nutritional change. Various knockout and transgenic overexpression animal models have demonstrated the important roles IGFs play in embryonic development and somatic growth (17-21). Circulating IGFs usually form complexes with binding proteins (IGFBP1-6). The IGFBPs not only provide an IGF hormone reservoir and regulate their bioavailability by forming IGFs/IGFBPs complexes, but also directly affect cell function via IGFs independent mechanisms (22-31). IGFBP-2 is the second most abundant IGF binding protein in the circulation, and contains a unique Arg-Gly-Asp (RGD) sequence, which can interact with cell surface integrin receptors (23). IGFBP2 has been shown to be a significant biomarker for several types of cancers (32-34), and circulating IGFBP2 levels have been highly correlated with pulmonary fibrosis disease progression and treatment (35).

In this study, the present inventors measured IGFBP2 and total IGF1/2 levels in test and verification PAH cohorts and a healthy control cohort in order to evaluate the value of these proteins as diagnostic biomarkers for PAH and determine their relationships with PAH progression and severity.

### Materials and Methods

Study Cohorts. Each study cohort was approved by the Institutional Review Board at its respective participating institution, including Johns Hopkins University, Cincinnati Children's Hospital Medical Center, and Vanderbilt University, with all subjects' consent.

PAH Patient Cohorts. The Johns Hopkins Pulmonary Hypertension (JHPH) cohort is an independent cohort of adult patients that was enrolled through the JHPH Program (n=127) (Table 1). MPH maintains the registry, and the inclusion criteria, exclusion criteria, and clinical assessments and therapy were published previously (36). Briefly, patients 18 years of age or older who were diagnosed with PAH by right heart catheterization and evaluated between Jan. 1, 2007 and Dec. 31, 2012 were included and classified into etiological groups on the basis of current guidelines defined by Fifth World Symposium on Pulmonary Hypertension (3). The diagnosis of CTD was based on meeting one of the following definitions (37): the American College of Rheumatology criteria, the presence of at least three of five features of the CREST syndrome (calcinosis, Raynaud's phenomenon, esophageal dysmotility, sclerodactyly, and telangiectasia), or definite Raynaud's phenomenon and the presence of a specific systemic sclerosis-related autoantibody.

National Biological Sample and Data Repository for Pulmonary Arterial Hypertension, or PAH Biobank (PAHB) is a NHLBI funded resource of 2900 WHO Group 1 PAH patient biological samples, genetic data, and clinical data enrolled from 38 US Centers (www.pahbiobank.org). Under a PAHBiobank approved protocol, the present inventors analyzed PAHBiobank enrollees with a cardiac catheterization within 6 months of enrollment (N=225).

Control Cohorts. The control serums from healthy adult volunteers were collected in three independent research centers: Johns Hopkins Pulmonary Center, Johns Hopkins Anesthesia Safety Study and Vanderbilt University Medical Center. A total of 128 volunteers participated, and their demographic information is summarized in Table 2.

IGF1, IGF2 and IGFBP2 measurements in serum. Total IGF1, IGF2, and IGFBP2 serum levels were measured using commercial ELISA kits (R&D, Cat #DG100, Human IGF-I Quantikine ELISA Kit; Cat #DG200, Human IGF-II Quantikine ELISA Kit; Cat #DGB200, Human IGFBP-2 Quantikine ELISA Kit). For IGF1 and 2 measurements, serum samples were pre-treated as instructed by the manufacturer to disassociate them from their binding proteins before the assay. All assays required the serum samples to be properly diluted (dilution factors were 100, 2000 and 50 respectively). All ELISA procedures and data analysis were performed according to manufacturer instructions.

Statistical Analysis. The present inventors used the JHPH cohort as the test cohort and the PAHB as the verification cohort. IGF1, IGF2, and IGFBP2 levels, demographic and functional test data are presented as median and interquartile range (IQR), or mean and standard deviation, number and percentage, where appropriate. The present inventors studied the association of logarithmically transformed serum IGFBP2, and IGFs with various clinical measures using unadjusted tests: spearman's rank correlation test for continuous variables and Kruskal Wallis test for categorical variables; using age and sex adjusted regression: linear for continuous variables, or logistic for dichotomous variables. The present inventors examined the association of IGFBP2, IGFs levels dichotomized at the median with mortality using unadjusted Kaplan-Meier analysis and Cox proportional Hazard regression analysis adjusted for age, gender, NYHA-FC, hemodynamics (RAP, PAP, PVR), PAH type and 6MWD. A p-value less than 0.05 was considered statistically significant. Statistical analysis was performed using STATA (Version 15, StataCorp LLC, College Station, Tex.) and MedCalc statistical software version 18.11.3 (2019 version; MedCalc Software, Ostend, Belgium).

### Results

Test cohort (JHPH cases). The present inventors used the JHPH cohort as a test cohort for the present hypothesis about the roles of IGF axis proteins as PAH biomarkers. The demographic information and clinical characteristics of the JHPH cohort (N=127) are summarized in Table 1. The serum samples of all patients were obtained at a single time point (enrollment). The median age was 62 years old, and 84% were women. 36% of the patients were diagnosed with IPAH and 64% with PAH associated with connective tissue disease (APAH-CTD). The overall mortality was 46%, with 58 deaths during the follow-up period of 5 years.

As shown in Table 3, the circulating IGFBP2 concentration was significantly increased in PAH compared with healthy control subjects (median 350.9 ng/ml vs 170 ng/ml, p<0.0001), though no significant differences were observed for IGF1 (median 67.0 vs 64.7 ng/ml, p=0.1) and IGF2 (347.8 vs 340.9 ng/ml, p=0.6). The present inventors also calculated the molar concentrations of IGF1 and 2, and IGFBP2 and calculated the IGF 1, 2, total IGFs to IGFBP2 ratios as measures of free IGFs (51) compared to controls. IGF to IGFBP2 molar ratios decreased significantly for IGF1/IGFBP2 (median 1.0 vs 1.7 nmol/L, p<0.0001), IGF2/IGFBP2 (median 5.1 vs 10.3 nmol/L, p<0.0001), or total IGFs/IGFBP2 (median 6.1 vs 11.9 nmol/L, p<0.0001).

Verification cohort (PAHB cases). A larger cohort from NHLBI PAHbiobank (PAHB, N=225) was used as the verification cohort. The demographic data is similar to the data from MPH cohort, and is summarized in Table 1. The median age was 57 years old, and 79% were female, 39% with IPAH and 52% with APAH. Overall mortality was 48.9%, with 110 deaths during the 5 year follow-up period.

In the PAHB cohort, circulating IGFBP2 concentration was significantly increased compared with healthy control subjects (median 500.1 vs 170.2 ng/ml, p<0.0001). IGF1 was also significantly increased (median 78.2 vs 64.7 ng/ml, p<0.0001), but no significant difference existed for IGF2 (393.0 vs 340.9 ng/ml, p=0.2). When IGF to IGFBP2 molar ratios were determined, IGF1/IGFBP2 (median 0.8 vs 1.7 nmol/L, p<0.0001), IGF2/IGFBP2 (median 3.3 vs 10.3 nmol/L, p<0.0001), or total IGF/IGFBP2 (median 4.0 vs 11.9 nmol/L, p<0.0001) were all significantly decreased.

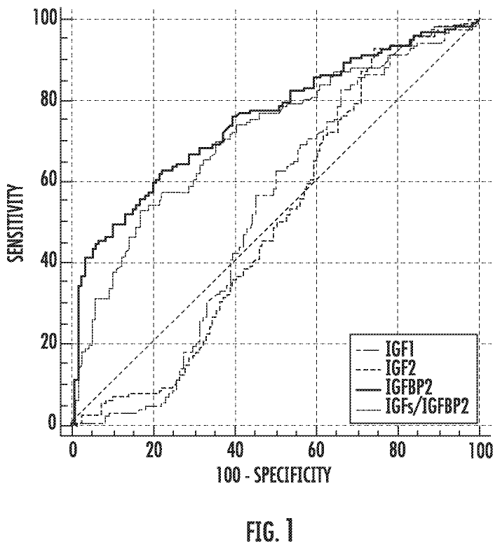

IGFBP2 discriminates PAH from Healthy Controls. In order to evaluate whether IGF axis proteins are able to discriminate patients with PAH from healthy controls, the present inventors generated ROC curves for IGF1, IGF2, and IGFBP2 as well as the molar ratio of IGFs to IGFBP2, using their values from JHPH and control cohorts. As shown in FIG. 1, among the four ROC curves, IGFBP2 was the best performer with an AUC of 0.76 (95% confidence interval [CI] 0.698-0.808, P<0.0001). A serum IGFBP2 cut-off value was established as 7.3 nmol/L to distinguish PAH from controls by Youden analysis. This cut-off value had a sensitivity and specificity for PAH of 62.2% and 78.5% respectively. The performance of this cut-off value was then tested with the present verification PAHB cohort and healthy controls. In this validation analysis with a case prevalence of 63%, the test performed well in discriminating PAH, with positive and negative predictive values of 85% and 77% respectively. For IGFBP2 values less than 7.3 nmol/L, the likelihood ratio for the presence of PH was 0.19 (95% confidence interval [CI] 0.14-0.26), while for values of 7.3 or greater, the likelihood ratio was 4.0 (95% CI 2.83-5.65).

IGF axis biomarkers and PAH Severity. The present inventors determined the relationship between serum IGF1, IGF2, and IGFBP2 and invasive resting hemodynamics, exercise tolerance assessed by 6 minutes walk distance (6MWD), and functional class. Using Spearman correlation, there was modest correlation of serum IGFBP2 with hemodynamics, specifically right atrial pressure (RAP) (r=0.133, P=0.049) in the PAHB cohort only. Significant negative correlation of IGFBP2 with 6MWD was observed in both JHPH (r=−0.233, p=0.018) and PAHB (r=−0.316, p<0.0001) cohorts (Table 4). Using linear regression analysis adjusted for age and gender, each log-unit increase in IGFBP2 was associated with a 9 mmHg higher mean pulmonary artery pressure (PAP) in the JHPH cohort (coefficient 9.103, p=0.026); In the PAHB cohort, each log-unit increase in IGFBP2 was associated with a 1 mmHg higher mean RAP (coefficient 1.328, p=0.041). Each log-unit increase of IGFBP2 was associated with a 52 m decrease in 6MWD (coefficient −51.669, p=0.016) (Table 5). There was no significant association between any of the IGF proteins with functional class. When the present inventors studied subtypes of PAH, IGFBP2 was significantly associated with APAH-CTD in both JHPH and PAHB cohorts (rank sum p<0.0001 for both cohorts); using logistic regression analysis after adjusting for age and gender, the significant association was only found in the PAHB cohort (coefficient 0.898, p=0.001) (Table 5).

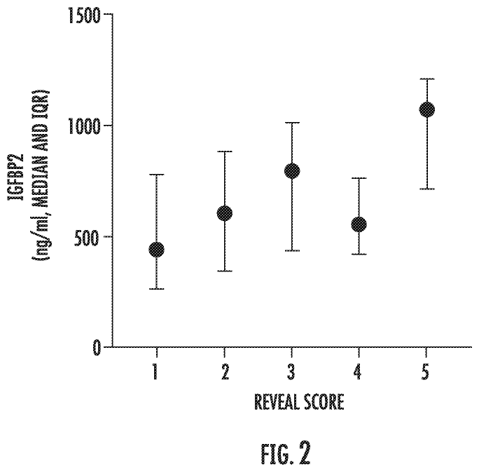

Serum IGF axis proteins and survival in PAH. After adjustment for age and gender, logistic regression analysis demonstrated that IGFBP2 was significantly associated with mortality in both JHPH (coefficient 2.86, p<0.0001) and PAHB (coefficient 1.18, p<0.0001, Table 5) cohorts. There was also negative association between mortality and IGF2 in PAHB only (coefficient −0.72, p=0.016). A validated predictive algorithm for 1-year survival was used to calculate the REVEAL (38,39) in the PAHB cohort. Using Kruskal Wallis, IGF2 and IGFBP2 were both significantly associated with an increasing REVEAL score (rank sum p=0.047 and p<0.0001 respectively). The plot of REVEAL risk category and IGFBP2 levels demonstrated a clear dose response relationship (FIG. 2).

The present inventors assessed the relationship between IGF axis protein levels and mortality using Kaplan-Meier analysis in JHPH and PABH cohorts. Elevated IGFBP2 levels above the median value were associated with a significantly increased risk of death, with an unadjusted hazard ratio of 2.97 (95% CI, 1.77-5.3; p=0.0001 by log-rank test) (FIG. 2A) in JHPH. However, neither IGF1 nor IGF2 was significantly associated with mortality in the JHPH cohort (p=0.82 for IGF1; p=0.23 for IGF2). Decreased IGFs/IGFBP2 molar ratio values below the median value were associated with an increased risk of death, with an unadjusted hazard ratio of 2.49 (95% CI, 1.46-4.26; p=0.001 by log-rank test), similar to that seen for IGFBP2 alone (FIG. 2B).

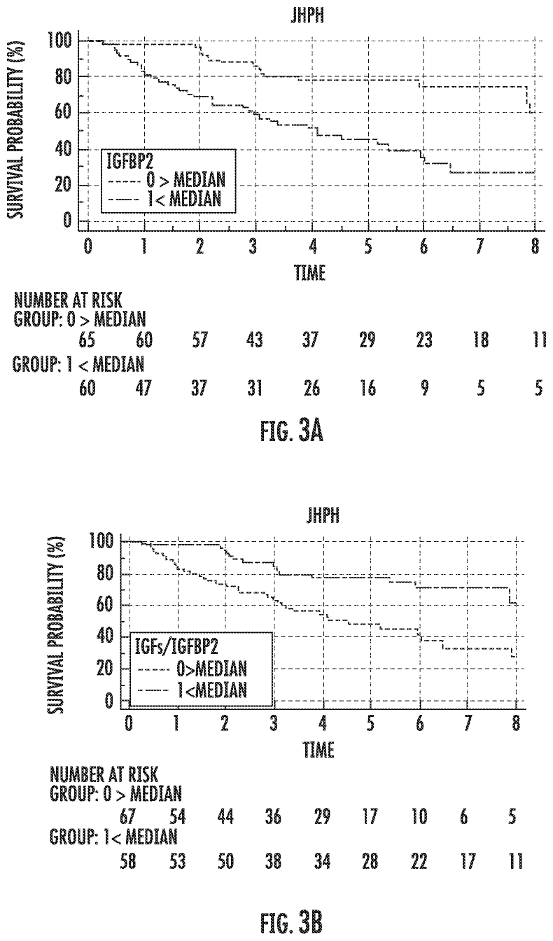

In the PAHB cohort, similar results were observed with IGFBP2 levels above the median value significantly associated with an increased risk of death, with an unadjusted hazard ratio of 4.13 (95% CI, 2.12-6.66; p<0.0001 by log-rank test) (FIG. 3A). In addition, decreased IGFs/IGFBP2 ratio values below the median were associated an increased risk of death, with an unadjusted hazard ratio of 7.29 (95% CI, 3.04-9.59; p<0.0001 by log-rank test) (FIG. 3B). Decreased concentrations of both IGF1 and IGF2 were also significantly associated with risk of death (HR 2.56, 95% CI, 1.41-4.42; p=0.002 for IGF1; HR 3.08, 95% CI, 1.64-5.13; p=0.0002 for IGF2).

The present inventors constructed a Cox multivariable proportional hazard model, adjusted for significant clinical variables: age, gender, NYHA-FC, hemodynamics (RAP, PAP, PVR), PAH type and 6MWD to examine the relationship between IGF axis proteins and survival. In the JHPH cohort, increased serum IGFBP2 predicted survival, with a hazard ratio [HR] of 5.29 (95% CI, 1.61-17.40; p=0.006). When the same model was applied to the PAHB cohort, the model performed well with a HR 4.12 (95% CI, 1.64-10.37; p=0.003).

### Discussion

In the current study, the present inventors tested and verified that IGFBP2 is markedly increased in PAH using 2 different PAH cohorts, a Johns Hopkins PAH cohort and a multicenter PAH cohort (NHLBI PAHBiobank). Using these cohorts, the present inventors demonstrate that IGFBP2 is significantly associated with PAH, survival and disease severity (REVEAL score, 6MWD).

IGFBP2 is a member of a large family of six binding proteins for IGF1 and IGF2 (14-15). In general, circulating IGF1 and 2 are bound to IGFBPs as free IGF is rapidly degraded. IGFBPs prevent IGF degradation and facilitate delivery of IGFs to the IGF cell surface receptors to trigger an essential IGF growth signal (14-16), however, some IGFBPs, including IGFBP2, have been shown to stimulate cell growth in an IGF independent manner (28, 31).

Circulating IGFBPs have also been associated with other cardiopulmonary diseases (35, 38-41). For example, IGFBP1 was one of several proteins identified from a proteomics screen of adult PAH patients with a high REVEAL score predicting a higher mortality risk (38). In adults with pulmonary fibrosis, another disease process that often complicates the collagen vascular diseases associated with PAH, IGFBP2 was found to be increased and was modulated by pulmonary fibrosis treatment (35,39). Circulating IGFBP7 was shown to be significantly associated with cardiac diastolic function in a reanalysis of the RELAX trial (41). Finally, the present inventors found that IGFBP2 was significantly elevated in PAH using two different PAH cohorts. The mechanisms driving elevated IGFBP2 expression at this point are unclear but appear to be unrelated to IGF1 and IGF2 concentrations.

In contrast to increased IGFBP2 in PAH patients, IGF1 and IGF2 in general were decreased and the molar ratio of IGF to IGFBP ratios, which better reflected IGF activity, were decreased in every cohort the present inventors examined, either individually or combined with IGF1 and 2 together. Compared to IGFBP2 alone, the ability of IGF1, IGF2 or IGF1+IGF2 to IGFBP molar ratio to distinguish subjects with PAH from healthy controls was less powerful. The correlation of these markers with hemodynamics and mortality were sporadically found in some cohorts, but none of them as consistent as IGFBP2. A similar pattern was found in patients with pulmonary fibrosis: IGF1 and IGF2 levels were significantly decreased, while IGFBP2 was strikingly increased (35,39). Therefore, relative IGF deficiency may be a common pathobiologic pathway of severe pulmonary vascular disease, but IGFBP2 may serve as a more important indicator of disease severity.

IGFBP2 is overexpressed in many tumors, and IGFBP2 expression levels are highly correlated with grade of malignancy and poor tumor differentiation (32-34). Recent studies have found that IGFBP2 is an essential component for maintaining ex vivo expansion of hematopoietic stem cells (HSC) (42,43). Although the mechanism is still unknown, IGFBP2 downregulates phosphatase and tensin homolog deleted on chromosome 10 (PTEN), a critical cell cycle inhibitor both in vivo and in vitro (43,44). Most importantly, a genetic mouse model with conditional knockout of PTEN in smooth muscle cells demonstrated spontaneous development of pulmonary hypertension, with increasing Akt activity in major vessels, heart, and lungs, and widespread medial SMC hyperplasia with vascular remodeling (45). In the present study, the present inventors found IGFBP2 was increased significantly in all PAH cohorts. It is intriguing to hypothesize that IGFBP2 may contribute to PAH pathogenesis through downregulation of PTEN and its underlying regulatory mechanism.

Low IGF1 levels are associated with increased risk for CVD and CVD mortality (46,47), suggesting that IGF1 plays an important role in cardiovascular diseases. The Framingham Heart Study demonstrated that lower IGF1 levels were independently associated with all-cause mortality (48). This observation has led to trials of growth hormone therapy with equivocal results (50,51). IGFBP2 plays an important role in the IGF growth axis, with elevated levels associated with lower fasting insulin and fasting glucose, but with greater mortality in older adults and in patients with dilated cardiomyopathy (52,53). Therefore, in large part, this IGF1 and IGFBP2 dysregulation serves as a negative growth signal. This is supported by animal data where transgenic IGFBP2 mouse overexpression with a ubiquitous CMV promoter resulted in decreased body mass and muscle weight (17,19). Of interest, IGFBP2 overexpression had a greater effect on somatic growth inhibition in females than males (19). Thus, the interplay and dysregulation of IGF1 and IGFBP2 may be important in explaining the increased female gender bias in PAH and why the disease is more severe in men.

In conclusion, IGFBP2 is a potential new PAH biomarker that, it associated with disease severity and survival and provides valuable clinical prognostic information. This study adds to a body of literature that, taken together, suggests IGFBP2 may contribute to PAH development through suppressing PTEN and/or cardiovascular metabolic effects. An improved understanding of this new pathway may support future development of novel therapeutic targets for PAH.

TABLE 1
Demographics and Characteristics of Pulmonary Arterial Hypertension Cohorts

| | JHPH Cohort | | | PAHB Cohort | | |
|---|---|---|---|---|---|---|
| n= | 127 | | | 225 | | |
| Age (years) | 62 (50-69) | | | 57 (45-68) | | |
| Female (n, %) | 107 (84%) | | | 177 (79%) | | |
| Race-EA, AA other n (%) | 95/20/12 (75%/16%/9%) | | | 187/28/10 (83%/12%/5%) | | |
| IPAH/APAH/Other, n, (%) | 46/81/0 (36%/64%/0%) | | | 87/118/20 (39%/52%/9%) | | |
| 6 MWD (m) | 376 | | 297-454 | 296 | | 183-402 |
| NYHA-FC | n = 127 | | % | n = 225 | | % |
| | I | (13) | 10% | I | (7) | 3% |
| | II | (53) | 42% | II | (53) | 24% |
| | III | (60) | 47% | III | (84) | 37% |
| | IV | (0) | 0% | IV | (13) | 6% |

| | | | | |
|---|---|---|---|---|
| | No data (0) | 1% | No data (68) | 30% |
| Laboratory Chemistries | | | | |
| NT-proBNP (pg/ml) | 779.5 | (249.5-2826.6) | 1254.1 | (365.6-4080.8) |
| Hemodynamics | | | | |
| RAP (mmHg) | 7 | (4-9) | 9 | (6-14) |
| mPAP (mmHg) | 39 | (29-50) | 48 | (40-56) |
| PCWP (mmHg) | 10 | (7-12) | 11 | (8-13) |
| PVR (WU) | 6.4 | (3.4-10.3) | 9.1 | (6.4-13.5) |
| CO (L min−1) | 4.4 | (3.7-5.5) | 4 | (3.2-4.9) |
| CI (L min⁻¹/m⁻²) | 2.5 | (2.1-3.1) | 2.2 | (1.8-2.9) |

Data expressed as median and IQR number (n), percentage (%) or range as indicated.
Definitions of abbreviations:
EA = European American,
AA = African American,
RAP = right atrial pressure,
mPAP = mean pulmonary artery pressure,
PCWP = pulmonary capillary w edge pressure,
PVR = pulmonary vascular resistance,
WU = Wood units,
CO = Cardiac output,
CI = Cardiac index,
NYHA-FC = New York Heart Associated-Functional Class,
6 MWD = 6 Minute Walk Distance,
NT-proBNP = N-terminal pro-brain natriuretic peptide,
JHPH = Johns Hopkins Pulmonary Hypertension,
PAHB = Pulmonary Arterial Hypertension Biobank

TABLE 2
Demographics of All Cohorts

| | Control Cohort | JHPH Cohort | PAHB Cohort |
|---|---|---|---|
| n= | 128 | 127 | 225 |
| Age (years) | 43 (3.3-57) | 62 (50-69) | 57 (45-68) |
| Female (n, %) | 96 (75%) | 107 (84%) | 177 (79%) |
| Race | | | |
| EA | 105 (82%) | 95 (75%) | 187 (83%) |
| AA | 15 (12%) | 20 (16%) | 26 (12%) |
| Other | 8 (6%) | 12 (9%) | 10 (5%) |

TABLE 3
Circulating IGF Axis Proteins Levels

| | Control, N = 128 | PAHB, N = 225 | JHPH, N = 127 |
|---|---|---|---|
| | mean ± SD | mean ± SD, p-value* | mean ± SD, p-value |
| | (Median; Range) | (Median; Range) | (Median; Range) |
| IGF1 (ng/ml) | 69.4 ± 34.4 | 89.2 ± 50.2, p < 0.0001 | 72.9 ± 30.4, p = 0.1 |
| | (64.7; 18.3-99.6) | (78.2; 13.7-486.6) | (67.0, 24.3-222.8) |
| IGF2 (ng/ml) | 385.0 ± 171.6 | 398.4 ± 178.3, p = 0.2 | 374.5 ± 121.9, p = 0.6 |
| | (340.9; 56.6-994.5) | (393.0; 55.4-963.4) | (347.8; 56.1-908.1) |
| IGFBP2 (ng/ml) | 207.3 ± 180.5 | 630.5 ± 419.5, p < 0.0001 | 419.8 ± 307.4, p < 0.0001 |
| | (170.2; 55.6-1806) | (500.1; 103.6-2144) | (350.9; 55.5-1869) |
| IGF1 (nmol/L) | 9.1 ± 4.5 | 11.7 ± 6.6, p < 0.0001 | 9.5 ± 4.0, p = 0.1 |
| | (8.5; 2.4-26.1) | (10.2; 1.8-63.6) | (8.8., 3.2-29.1) |
| IGF2 (nmol/L) | 51.5 ± 23.0 | 53.3 ± 23.9, p = 0.2 | 50.1 ± 16.3, p = 0.6 |
| | (45.6; 7.6-133.1) | (52.6; 7.5-129) | (46.6; 7.5-121.6) |
| IGFBP2 (nmol/L) | 5.7 ± 5.0 | 17.5 ± 11.2, p < 0.0001 | 11.7 ± 8.5, p < 0.0001 |
| | (4.7; 1.5-50.2) | (13.9; 2.9-59.6) | (9.7; 1.5-51.9) |
| IGF1/IGFBP2 molar ratio | 2.3 ± 1.8 | 1.0 ± 1.0, p < 0.0001 | 1.5 ± 1.6, p < 0.0001 |
| | (1.7; 0.1-10.1) | (0.8; 0.1-5.9) | (1.0; 0.1-12.4) |
| IGF2/IGFBP2 molar ratio | 12.6 ± 9.5 | 5.0 ± 5.4, p < 0.0001 | 7.4 ± 6.5, p < 0.0001 |
| | (10.3; 0.4-58.3) | (3.3; 0.1-44.0) | (5.1; 0.4-39.0) |
| IGF (1 + 2)/IGFBP2 molar ratio | 14.9 ± 11.1 | 6.0 ± 6.2, p < 0.0001 | 8.9 ± 7.8, p < 0.0001 |
| | (11.9; 0.5-68.4) | (4.0; 0.3-50.0) | (6.1; 0.5-51.4) |

*p values were calculated by t-test compared to corresponding control values.

TABLE 4
Associations (Unadjusted) between IGF axis proteins and clinical variables

| | JHPH | | | | | | PAHB | | | | | |
|---|---|---|---|---|---|---|---|---|---|---|---|---|
| | IGF1 | | IGF2 | | IGFBP 2 | | IGF1 | | IGF2 | | IGFBP 2 | |
| Spearman | | | | | | | | | | | | |
| Rank | Spearman | p- | Spearman | p- | Spearman | p- | Spearman | p- | Spearman | p- | Spearman | p- |
| Correlation | Rho | value | Rho | value | Rho | value | Rho | value | Rho | value | Rho | value |
| Age | [symbol] | [symbol] | 0.114 | [symbol] | [symbol] | [symbol] | [symbol] | [symbol] | [symbol] | [symbol] | [symbol] | [symbol] |
| BSA | [symbol] | 0.511 | [symbol] | 0.825 | [symbol] | [symbol] | 0.102 | [symbol] | [symbol] | 0.199 | [symbol] | [symbol] |
| SixMinWalkDist | 0.166 | 0.079 | −0.03 | 0.749 | [symbol] | [symbol] | −0.059 | [symbol] | [symbol] | 0.334 | [symbol] | [symbol] |
| (meters) | | | | | | | | | | | | |
| Cardiac output | [symbol] | 0.818 | [symbol] | 0.254 | 0.1 | [symbol] | [symbol] | 0.4 | [symbol] | 0.828 | [symbol] | [symbol] |
| Cardiac index | [symbol] | 0.545 | [symbol] | [symbol] | [symbol] | 0.769 | −0.111 | 0.104 | [symbol] | 0.267 | −0.039 | 0.572 |
| PVP | 0.097 | 0.307 | [symbol] | [symbol] | [symbol] | [symbol] | [symbol] | 0.414 | [symbol] | 0.588 | 0.041 | 0.0558 |
| mPAP | 0.111 | 0.215 | −0.035 | 0.034 | 0.112 | 0.212 | 0.063 | 0.314 | [symbol] | 0.407 | −0.047 | 0.486 |
| mRAP | [symbol] | 0.77 | 0.017 | [symbol] | [symbol] | [symbol] | 0.097 | [symbol] | −0.015 | 0.825 | [symbol] | [symbol] |
| mPCWP | −0.059 | 0.514 | 0.111 | 0.145 | [symbol] | [symbol] | 0.013 | 0.841 | −0.004 | [symbol] | −0.064 | 0.342 |
| Kruskal | p-value | | p-value | | p-value | | p-value | | p-value | | p-value | |
| Wallis Test | | | | | | | | | | | | |
| NYHA FC | 0.09 | | 0.119 | | 0.13 | | 0.315 | | 0.722 | | 0.134 | |
| REVEAL | | | | | | | 0.415 | | [symbol] | | [symbol] | |
| APAH Collagen | −0.0001 | | 0.001 | | −0.0001 | | 0.685 | | 0.306 | | [symbol] | |
| Vascular | | | | | | | | | | | | |
| Mortality | 0.409 | | 0.409 | | [symbol] | | [symbol] | | 0.001 | | [symbol] | |
| Logistric | | p- | | p- | | p- | | p- | | p- | | p- |
| Regression | Coefficient | value | Coefficient | value | Coefficient | value | Coefficient | value | Coefficient | value | Coefficient | value |
| Gender | −2.835 | 0.018 | 1.947 | 0.107 | [symbol] | [symbol] | −0.266 | 0.427 | [symbol] | [symbol] | 0.04 | 0.874 |

[symbol]

indicates data missing or illegible when filed

TABLE 5
Associations (adjusted for age and gender) between IGF axis proteins and clinical variables

| | JHPH | | | | | | PAHB | | | | | |
|---|---|---|---|---|---|---|---|---|---|---|---|---|
| | IGF1 | | IGF2 | | IGFBP2 | | IGF1 | | IGF2 | | IGFBP2 | |
| Linear Regression | coefficient | p-value | coefficient | p-value | coefficient | p-value | coefficient | p-value | coefficient | p-value | coefficient | p-value |
| BSA | −0.228 | 0.15 | 0.088 | 0.549 | −0.144 | 0.058 | 0.028 | 0.415 | 0.355 | [symbol] | [symbol] | [symbol] |
| SixMinWalkDist | [symbol] | 0.633 | 0.116 | 0.999 | [symbol] | 0.16 | [symbol] | [symbol] | 8.728 | [symbol] | [symbol] | [symbol] |
| (meters | | | | | | | | | | | | |
| Cardiac output | [symbol] | 0.545 | 1.277 | 0.19 | [symbol] | 0.554 | 0.012 | [symbol] | [symbol] | 0.775 | [symbol] | [symbol] |
| Cardiac index | −9.047 | 0.062 | 4.081 | 0.386 | 1.284 | 0.605 | −0.051 | 0.691 | −0.081 | 0.461 | [symbol] | 0.634 |
| PDR | 2.15 | 0.508 | −1.681 | [symbol] | 2.811 | 0.07 | 0.488 | 0.519 | [symbol] | [symbol] | [symbol] | [symbol] |
| mPAP | 5.382 | 0.532 | −6.841 | 0.4 | 9.198 | 0.026 | −0.183 | 0.916 | 0.013 | 0.993 | 0.749 | 0.588 |
| mRAP | 0.217 | 0.016 | −2.122 | 0.404 | 1.251 | 0.33 | 0.997 | 0.216 | −0.119 | [symbol] | [symbol] | [symbol] |
| mPCWP | −1.484 | 0.471 | −1.884 | 0.142 | 0.207 | 0.835 | 0.321 | 0.502 | −0.057 | 0.888 | [symbol] | [symbol] |
| Logistic Regression | coefficient | p-value | coefficient | p-value | coefficient | p-value | coefficient | p-value | coefficient | p-value | coefficient | p-value |
| NYHA-FC | −2.211 | 0.097 | −1.613 | 0.209 | 0.783 | 0.22 | 0.261 | 0.431 | 0.009 | 0.975 | 0.374 | 0.186 |
| APAH-Colalgen | −0.77 | 0.533 | 0.513 | 0.707 | 1.165 | 0.099 | 0.539 | 0.086 | −0.009 | 0.974 | [symbol] | [symbol] |
| Vascular | | | | | | | | | | | | |
| Mortality | [symbol] | [symbol] | −8.823 | 0.516 | [symbol] | [symbol] | −0.567 | 0.124 | [symbol] | [symbol] | [symbol] | [symbol] |

[symbol]

indicates data missing or illegible when filed

### REFERENCES

1. Humbert M, Gerry Coghlan J, Khanna D. Early detection and management of pulmonary arterial hypertension. Eur Respir Rev 2012; 21:306-312.
2. Rubin L J. Pulmonary arterial hypertension. Proc Am Thorac Soc 2006; 3:111-115.
3. Simonneau G, Montani D, Celermajer D S, Denton C P, Gatzoulis M A, Krowka M, Williams P G, Souza R. Haemodynamic definitions and updated clinical classification of pulmonary hypertension. Eur Respir J. 2019; 53:1801913.
4. Romberg E. Ueber Sklerose der Lungen arterie. Dtsch Arch Klin Med 1891; 48:197-206.
5. Hassoun P M, Mouthon L, Barbera'J A, Eddahibi S, Flores S C, Grimminger F, Jones P L, Maitland M L, Michelakis E D, Morrell N W, et al. Inflammation, growth factors, and pulmonary vascular remodeling. J Am Coll Cardiol 2009; 54(1, Suppl):S10-S19.
6. Budhiraja R, Tuder R M, Hassoun P M. Endothelial dysfunction in pulmonary hypertension. Circulation 2004; 109:159-165.
7. Thenappan T, Ormiston M L, Ryan J J, Archer S L. Pulmonary arterial hypertension: pathogenesis and clinical management. BMJ. 2018; 360:j5492. doi: 10.1136/bmj.j5492.
8. Thenappan T, Shah S J, Rich S, Gomberg-Maitland M. A USA-based registry for pulmonary arterial hypertension: 1982-2006[eng.]. Eur Respir J 2007; 30:1103-10. doi:10.1183/09031936.00042107 pmid:17804449.
9. McLaughlin V V, Archer S L, Badesch D B, et al. American College of Cardiology Foundation Task Force on Expert Consensus Documents American Heart Association American College of Chest Physicians American Thoracic Society, Inc. Pulmonary Hypertension Association. ACCF/AHA 2009 expert consensus document on pulmonary hypertension a report of the American College of Cardiology Foundation Task Force on Expert Consensus Documents and the American Heart Association developed in collaboration with the American College of Chest Physicians; American Thoracic Society, Inc.; and the Pulmonary Hypertension Association. J Am Coll Cardiol 2009; 53:1573-619. doi:10.1016/j.jacc.2009.01.004 pmid:19389575.
10. Galiè N, Humbert M, Vachiery J L, et al. 2015 ESC/ERS Guidelines for the diagnosis and treatment of pulmonary hypertension: The Joint Task Force for the Diagnosis and Treatment of Pulmonary Hypertension of the European Society of Cardiology (ESC) and the European Respiratory Society (ERS): Endorsed by: Association for European Paediatric and Congenital Cardiology (AEPC), International Society for Heart and Lung Transplantation (ISHLT). Eur Heart J 2016; 37:67-119. doi:10.1093/eurheartj/ehv317 pmid:26320113.
11. Nir A, Lindinger A, Rauh M, Bar-Oz B, Laer S, Schwachtgen L, Koch A, Falkenberg J, Mir T S. NT-pro-B-type natriuretic peptide in infants and children: reference values based on combined data from four studies. Pediatr Cardiol 2009; 30:3-8.
12. Fritz J S, Blair C, Oudiz R J, Dufton C, Olschewski H, Despain D, Gillies H, Kawut S M. Baseline and follow-up 6-min walk distance and brain natriuretic peptide predict 2-year mortality in pulmonary arterial hypertension. Chest 2013; 143:315-323.
13. Panagopoulou V, Deftereos S, Kossyvakis C, Raisakis K, Giannopoulos G, Bouras G, Pyrgakis V, Cleman M W. NTproBNP: an important biomarker in cardiac diseases. Curr Top Med Chem 2013; 13:82-94.
14. Hwa V, Oh Y, Rosenfeld R G. The insulin-like growth factor-binding protein (IGFBP) superfamily. Endocr Rev 1999; 20(6):761-87.
15. Haywood N J, Slater T A, Matthews C J, Wheatcroft S B. The insulin like growth factor and binding protein family: Novel therapeutic targets in obesity & diabetes. Mol Metab 2018; pii: S2212-8778(18)30893-7. doi: 10.1016/j.molmet.2018.10.008.
16. Collett-Solberg P F and Cohen P. Genetics, chemistry, and function of the IGF/IGFBP system. Endocrine 2000; 12(2):121-36.
17. Schneider M R, Lahm H, Wu M, Hoeflich A, Wolf E. Transgenic mouse models for studying the functions of insulin-like growth factor-binding proteins. FASEB J 2000; 14(5):629-40.
18. DeMambro V E, Clemmons D R, Horton L G, Bouxsein M L, Wood T L, Beamer W G, Canalis E, Rosen C J. Gender-specific changes in bone turnover and skeletal architecture in igfbp-2-null mice. Endocrinology. 2008; 149(5):2051-61. doi: 10.1210/en.2007-1068.
19. Silha J V and Murphy L J. Insights from insulin-like growth factor binding protein transgenic mice. Endocrinology. 2002; 143(10):3711-4.
20. Wolf E, Lahm H, Wu M, Wanke R, Hoeflich A. Effects of IGFBP-2 overexpression in vitro and in vivo. Pediatr Nephrol. 2000; 14(7):572-8.
21. Wood A W, Schlueter P J, Duan C. Targeted knockdown of insulin-like growth factor binding protein-2 disrupts cardiovascular development in zebrafish embryos. Mol Endocrinol. 2005; 19(4):1024-34.
22. Bach L A. What Happened to the IGF Binding Proteins? Endocrinology 2018; 159(2):570-578. doi: 10.1210/en.2017-00908.
23. Wang G K, Hu L, Fuller G N, Zhang W. An interaction between insulin-like growth factor-binding protein 2 (IGFBP2) and integrin alpha5 is essential for IGFBP2-induced cell mobility. J Biol Chem. 2006; 281(20):14085-91.
24. Jones J I, Gockerman A, Busby W H Jr, Wright G, Clemmons D R. Insulin-like growth factor binding protein 1 stimulates cell migration and binds to the alpha 5 beta 1 integrin by means of its Arg-Gly-Asp sequence. Proc Natl Acad Sci USA 1993; 90:10553-7.
25. Schedlich L J, Le Page S L, Firth S M, Briggs L J, Jans D A, Baxter R C. Nuclear import of insulin-like growth factor-binding protein-3 and −5 is mediated by the importin beta subunit. J Biol Chem 2000; 275:23462-70.
26. Xu Q, Li S, Zhao Y, Maures T J, Yin P, Duan C. Evidence that IGF binding protein-5 functions as a ligand-independent transcriptional regulator in vascular smooth muscle cells. Circ Res 2004; 94:E46-54.
27. Zhong Y, Lu L, Zhou J, Li Y, Liu Y, Clemmons D R, et al. IGF binding protein 3 exerts its ligand-independent action by antagonizing BMP in zebrafish embryos. J Cell Sci 2011; 124:1925-35.
28. Azar W J, Azar S H, Higgins S, Hu J F, Hoffman A R, Newgreen D F, et al. IGFBP-2 enhances VEGF gene promoter activity and consequent promotion of angiogenesis by neuroblastoma cells. Endocrinology 2011; 152:3332-42.
29. Zhu W, Shiojima I, Ito Y, Li Z, Ikeda H, Yoshida M, et al. IGFBP-4 is an inhibitor of canonical Wnt signalling required for cardiogenesis. Nature 2008; 454:345-9.
30. Oh Y, Muller H L, Lamson G, Rosenfeld R G. Insulin-like growth factor (IGF)-independent action of IGF-binding protein-3 in Hs578T human breast cancer cells. Cell surface binding and growth inhibition. J Biol Chem 1993; 268:14964-71.
31. Xi G, Wai C, DeMambro V, Rosen C J, Clemmons D R. IGFBP-2 directly stimulates osteoblast differentiation. J Bone Miner Res (2014) 29(11):2427-38.
32. Yoneyama T, Ohtsuki S, Honda K, Kobayashi M, Iwasaki M, Uchida Y, Okusaka T, Nakamori S, Shimahara M, Ueno T, Tsuchida A, Sata N, loka T, Yasunami Y, Kosuge T, Kaneda T, Kato T, Yagihara K, Fujita S, Huang W, Yamada T, Tachikawa M, Terasaki T. Identification of IGFBP2 and IGFBP3 As Compensatory Biomarkers for CA19-9 in Early-Stage Pancreatic Cancer Using a Combination of Antibody-Based and L C-M S/M S-Based Proteomics. PLoS One. 2016; 11(8):e0161009. doi: 10.1371/journal.pone.0161009.
33. Hur H, Yu E J, Ham I H, Jin H J, Lee D. Preoperative serum levels of insulin-like growth factor-binding protein 2 predict prognosis of gastric cancer patients. Oncotarget. 2017; 8(7):10994-11003.
34. Guo C, Lu H, Gao W, Wang L, Lu K, Wu S, Pataer A, Huang M, El-Zein R, Lin T, Roth J A, Mehran R, Hofstetter W, Swisher S G, Wu X, Fang B. Insulin-like growth factor binding protein-2 level is increased in blood of lung cancer patients and associated with poor survival. PLoS One. 2013; 8(9):e74973. doi: 10.1371/journal.pone.0074973
35. Mehrian-Shai R, Chen C D, Shi T, Horvath S, Nelson S F, Reichardt J K, Sawyers C L. Insulin growth factor-binding protein 2 is a candidate biomarker for PTEN status and PI3K/Akt pathway activation in glioblastoma and prostate cancer. Proc Natl Acad Sci USA. 2007; 104:5563-8.
36. Guiot J, Bondue B, Henket M, Corhay J L, Louis R. Raised serum levels of IGFBP-1 and IGFBP-2 in idiopathic pulmonary fibrosis. BMC Pulm Med. 2016; 16:86. doi: 10.1186/s12890-016-0249-6.
37. Damico R, Kolb T M, Valera L, Wang L, Housten T, Tedford R J, Kass D A, Rafaels N, Gao L, Barnes K C, et al. Serum endostatin is a genetically determined predictor of survival in pulmonary arterial hypertension. Am J Respir Crit Care Med 2015; 191:208-218.
38. Subcommittee for Scleroderma Criteria of the American Rheumatism Association Diagnostic and Therapeutic Criteria Committee. Preliminary criteria for the classification of systemic sclerosis (scleroderma). Arthritis Rheum 1980; 23:581-590.
39. Benza R L, Gomberg-Maitland M, Miller D P, Frost A, Frantz R P, Foreman A J, Badesch D B, McGoon M D: The REVEAL Registry risk score calculator in patients newly diagnosed with pulmonary arterial hypertension. Chest 2012, 141(2):354-362.
40. Benza R L, Miller D P, Gomberg-Maitland M, Frantz R P, Foreman A J, Coffey C S, Frost A, Barst R J, Badesch D B, Elliott C G et al: Predicting survival in pulmonary arterial hypertension: insights from the Registry to Evaluate Early and Long-Term Pulmonary Arterial Hypertension Disease Management (REVEAL). Circulation 2010, 122(2):164-172.
41. Rhodes C J, Wharton J, Ghataorhe P, Watson G, Girerd B, Howard L S, Gibbs J S R, Condliffe R, Elliot C A, Kiely D G, Simonneau G, Montani D, Sitbon O, Gall H, Schermuly R T, Ghofrani H A, Lawrie A, Humbert M, Wilkins M R. Plasma proteome analysis in patients with pulmonary arterial hypertension: an observational cohort study. Lancet Respir Med. 2017; 5:717-726. doi: 10.1016/S2213-2600(17)30161-3.
42. Guiot J, Henket M, Corhay J L, Louis R. Serum IGFBP2 as a marker of idiopathic pulmonary fibrosis. European Respiratory Journal 2015 46: PA3840; DOI: 10.1183/13993003.
43. Hoeflich A, David R, Hjortebjerg R. Current IGFBP-Related Biomarker Research in Cardiovascular Disease-We Need More Structural and Functional Information in Clinical Studies. Front Endocrinol (Lausanne). 201816; 9:388. doi: 10.3389/fendo.2018.00388.
44. Gandhi P U, Gaggin H K, Redfield M M, Chen H H, Stevens S R, Anstrom K J, Semigran M J, Liu P, Januzzi J L Jr. Insulin-Like Growth Factor-Binding Protein-7 as a Biomarker of Diastolic Dysfunction and Functional Capacity in Heart Failure With Preserved Ejection Fraction: Results From the RELAX Trial. JACC Heart Fail. 2016 November; 4(11):860-869. doi: 10.1016/j.jchf.2016.08.002.
45. Huynh H, Iizuka S, Kaba M, Kirak O, Zheng J, Lodish H F, Zhang C C.

Insulin-like growth factor-binding protein 2 secreted by a tumorigenic cell line supports ex vivo expansion of mouse hematopoietic stem cells. Stem Cells. 2008; 26(6):1628-35. doi: 10.1634/stemcells.2008-0064.
46. Huynh H, Zheng J, Umikawa M, Zhang C, Silvany R, lizuka S, Holzenberger M, Zhang W, Zhang CC. IGF binding protein 2 supports the survival and cycling of hematopoietic stem cells. Blood. 2011 22; 118(12):3236-43. doi: 10.1182/blood-2011-01-331876.
47. Perks C M, Vernon E G, Rosendahl A H, Tonge D, Holly J M. IGF-II and IGFBP-2 differentially regulate PTEN in human breast cancer cells. Oncogene. 2007; 26(40):5966-72.
48. Nemenoff R A, Simpson P A, Furgeson S B, Kaplan-Albuquerque N, Crossno J, Garl P J, Cooper J, Weiser-Evans M C. Targeted deletion of PTEN in smooth muscle cells results in vascular remodeling and recruitment of progenitor cells through induction of stromal cell-derived factor-1alpha. Circ Res. 2008; 102(9):1036-45. doi: 10.1161/CIRCRESAHA.
49. Burgers A M, Biermasz N R, Schoones J W, Pereira A M, Renehan A G, Zwahlen M, Egger M, Dekkers O M. Meta-analysis and dose-response metaregression: circulating insulin-like growth factor I (IGF-I) and mortality. J Clin Endocrinol Metab. 2011; 96(9):2912-20.
Laughlin G A, Barrett-Connor E, Criqui M H, Kritz-Silverstein D. The prospective association of serum insulin-like growth factor I (IGF-I) and IGF-binding protein-1 levels with all cause and cardiovascular disease mortality in older adults: the Rancho Bernardo Study. J Clin Endocrinol Metab. 2004; 89(1):114-20.
51. Roubenoff R, Parise H, Payette H A, Abad L W, D'Agostino R, Jacques P F, Wilson P W, Dinarello C A, Harris T B. Cytokines, insulin-like growth factor 1, sarcopenia, and mortality in very old community-dwelling men and women: the Framingham Heart Study. Am J Med. 2003; 115(6):429-435.
52. Isgaard J, Bergh C H, Caidahl K, Lomsky M, Hjalmarson A, Bengtsson B A. A placebo-controlled study of growth hormone in patients with congestive heart failure. Eur Heart J. 1998; 19(11): 1704-1711.
53. Osterziel K J, Strohm O, Schuler J, et al. Randomised, double-blind, placebo-controlled trial of human recombinant growth hormone in patients with chronic heart failure due to dilated cardiomyopathy. Lancet. 1998; 351(9111):1233-1237.
54. Hu D, Pawlikowska L, Kanaya A, Hsueh W C, Colbert L, Newman A B, Satterfield S, Rosen C, Cummings S R, Harris T B, Ziv E; Health, Aging, and Body Composition Study. Serum insulin-like growth factor-1 binding proteins 1 and 2 and mortality in older adults: the Health, Aging, and Body Composition Study. J Am Geriatr Soc. 2009 July; 57(7):1213-8.
55. Hassfeld S, Eichhorn C, Stehr K, Naegele H, Geier C, Steeg M, Ranke M B, Oezcelik C, Osterziel K J. Insulin-like growth factor-binding proteins 2 and 3 are independent predictors of a poor prognosis in patients with dilated cardiomyopathy. Heart. 2007 March; 93(3):359-60.
56. Russo V C, Azar W J, Yau S W, Sabin M A and Werther G A. IGFBP-2: The dark horse in metabolism and cancer. Cytokine Growth Factor Rev. 2015; 26:329-46.55.
57. Kwak I, Song S, Blum J L, Simmen RCM and Simmen F A. Enhancer- and Silencer-Like Sequences That Mediate Insulin-Like Growth Factor-Binding Protein-2 Gene Expression in Uterine Cells of Pregnancy. DNA AND CELL BIOLOGY. 2006; 25:6-18.
58. S.Pons and I. Torres-Aleman. Estradiol Modulates Insulin-Like Growth Factor I Receptors and Binding Proteins in Neurons from the Hypothalamus. Journal of Neuroendocrinology. 1993; 5:267-271.
59. 'mez GPC-G, Chowen J A and Garcia-Segura L M. Estradiol and Progesterone Regulate the Expression of Insulin-like Growth Factor-I Receptor and Insulinlike Growth Factor Binding Protein-2 in the Hypothalamus of Adult Female Rats. Journal of Neurobiology. 2000:269-281.

### Example 2: Insulin Like Growth Factors (IGFs) Play a Significant Role in Cardiopulmonary Function and May Play a Significant Role in the Pathobiology of PAH

This study sought to assess the diagnostic and prognostic value of IGF1, IGF2 and IGFBP2 in pediatric PAH.

Using an enzyme-linked immunosorbent assay, the present inventors evaluated serum total IGF1, IGF2, and IGFBP2 in a cohort of pediatric PAH patients (0-21 years) from the NHLBI PAH Biobank (N=175) and an age and gender matched cohort of healthy controls with no cardiopulmonary disease from Johns Hopkins (N=75). Serum protein IGF1, IGF2 and IGFBP2 concentrations and the molar ratio of total IGF/IGFBP2 as a measure of free IGF were analyzed with demographic, clinical and hemodynamic variables for relevance to pediatric PAH.

Serum IGFBP2 concentrations were significantly elevated in PAH patients compared to controls (264 vs 175 ng/mL; p=0.0004, 95% CI −109, −33). IGFBP2 could significantly discriminate PAH from control with an AUCROC of 0.64 (P<0.001). Total IGF/IGFBP2 was lower in pediatric PAH patients compared to controls (9.2 vs 16; p=0.0003; 95% CI −8.2, −2). IGF1 and total IGF/IGFBP2 ratio were significantly negatively correlated with pulmonary vascular resistance index, PVRi, (R=−0.479, P=0.003), while IGFBP2 concentrations were significantly positively correlated with PVRi (R=0.354, P=0.032). IGFBP2 was also negatively correlated with cardiac output (r=−0.481, p=0.008), while IGF1 was positively correlated with cardiac output (r=0.372, p=0.047). More importantly, IGFBP2 was negatively correlated with 6-minute walk distance (r=−0.576, p=0.004) while Total IGF/IGFBP2 was positively correlated with 6-minute walk distance (r=0.492, p=0.017). Higher median IGFBP2 levels were significantly associated with more intensive medical therapy, particularly need for multiple medications and IV or subcutaneous prostacyclin or prostacyclin analogues (median IGFBP2 358 ng/mL, IQR 232-503, p<0.0001). Finally, in logistic regression adjusted for age and gender, higher IGFBP2 and lower total IGF/IGFBP2 were significantly associated with mortality.

IGFBP2 concentrations are elevated in PAH patients and discriminates PAH patients from healthy controls. IGFBP2 is a novel prognostic marker for pediatric PAH with ability to distinguish more severe disease, including worse functional status, need for chronic infusion therapy, and survival. Total IGF/IGFBP2 ratios are lower compared to controls suggesting that there is a relative IGF deficiency in PAH patients. Considering the importance of IGF in regulating cardiac function and metabolism, the dysregulation of this axis may be an important mechanistic target in pediatric pulmonary arterial hypertension.

### Introduction

Pulmonary arterial hypertension (PAH) in children is a progressive and almost uniformly fatal disease characterized by sustained elevation of pulmonary arterial pressures and death from right ventricular failure (1). Pediatric pulmonary hypertension is a heterogenous disease which may be caused by development, prematurity, bronchopulmonary dysplasia, perinatal pulmonary vascular maladaptation, and other congenital or genetic abnormalities (1). The pathobiology of pulmonary hypertension is incompletely understood, but results in fixed vascular obstruction; endothelial dysfunction, seen in microvascular remodeling. The characteristic plexiform lesions, is thought to play a role in this hyper-proliferative state, which in turn significantly affects the adaptation and function of the right ventricle to the escalating pressure load caused by increasing pulmonary vascular resistance (2,3).

The growth and metabolic regulation of the pulmonary vasculature and right ventricle in pediatric pulmonary hypertension is incompletely understood, but there is a complex interaction of growth and metabolic factors involved (4). Insulin like growth factors (IGF1 and IGF2) play an essential role in normal cardiopulmonary development and function (5). IGF actions are mediated by the IGF1/IGF2 receptors; in turn, IGF receptor interactions are modulated by a large family of seven high-affinity IGF binding proteins (IGFBP1-7) (6), with IGFBP2 found in some studies of pulmonary disease (7). While IGF1 is the primary mediator of these actions, most IGF1 is bound to and stabilized by one of the IGF binding proteins, as free IGF1 is rapidly degraded. Prior proteomic analysis by Rhodes et al. (8) in adult PAH patients showed increased IGFBP1, and incorporation of IGFBP1 into a multi-protein panel was significantly associated with a worse REVEAL (Registry to Evaluate Early and Long Term Pulmonary Arterial Hypertension Disease Management) score (8). Circulating IGFBP2 was similarly increased in patients with pulmonary fibrosis, with circulating levels associated with treatment response (7). IGFBP1, IGFP2 and IGFBP7 have been shown to be predictive of worsening cardiovascular outcomes in patients with left sided heart failure (9-11). IGFBP7, in particular, was a prognostic biomarker for heart failure with reduced ejection fraction and showed a significant association with abnormal diastolic function (11).

With that background, this study examines the relationship of IGFBP2, and the proteins it binds, IGF1 and IGF2, as predictors of pediatric PAH severity.

### Materials and Methods

In this multicenter cross-sectional study of pediatric pulmonary hypertension, the present inventors identified and analyzed IGF1, IGF2, and IGFBP2 concentrations from a cohort of pediatric PAH patients with WHO group 1 pulmonary hypertension, and a cohort of age and gender-matched healthy controls.

Study Cohorts. NHLBI PAH Biobank patient enrollment protocol and informed consent has been approved by the Cincinnati Children's Hospital Medical Center Institutional Review Board. All cohorts have been approved by the Johns Hopkins University Institutional Review Board. Control samples from Johns Hopkins Hospital were collected after informed consent by Johns Hopkins investigators. All samples have been collected with informed consent.

Pulmonary Hypertension Patients. NHLBI PAH Biobank: The PAH Biobank is a NHLBI (HL105333) funded resource of biological samples, genetic data, and clinical data for the PAH research community, with 5 pediatric enrolling centers. De-identified biological samples and clinical data on enrollees (N=175, <21 years old, Table 1) were available for analysis. The PAH Biobank collects the PAH clinical severity parameters at enrollment including the WHO group 1 subgroup (specific to PAH), demographics and comorbidities, NYHA/WHO functional class, 6-minute walk distance (6MWD), drug therapy and right heart catheterization data. A subset of the PAH cohort had right heart catheterizations within one year (N=49) or within 6 months of enrollment (N=29) and were used for invasive cardiac hemodynamic analysis. Other measures of severity including further medical or surgical therapy, and mortality were collected prospectively.

Control Patients. As healthy controls, children (N=75, 1 month to 21 years, Table 1) presenting for elective surgery had serum obtained while under general anesthesia at the Johns Hopkins Children's Center. Samples were collected with Johns Hopkins IRB approval with informed consent and with patient assent where appropriate. None had pulmonary hypertension or lung disease.

Lab methods. All PAH patient and control samples were assayed for total (free and bound) IGF1 and IGF2, and IGFBP2 using commercial ELISAs (R & D Systems, Minneapolis, Minn. Cat #DGB200, Human IGFBP-2 Quantikine ELISA Kit; Cat #DG100, SG100, PDG100, Human IGF1; Cat # DG200, Human IGF2). The serum samples were diluted and assayed according to manufacturer instructions. Assays were analyzed using KC4 (Bio-Tek Instruments, Winooski, Vt.). All samples were assayed while blinded to clinical outcomes and only unblinded for statistical analysis.

Statistical Analysis. Total IGF1, IGF2, and IGFBP2 concentrations are presented as median and interquartile range (IQR). Demographic and functional data are presented as median and interquartile range, or median, percent, and range as appropriate. As IGF1, IGF2, and IGFBP2 concentrations were not normally distributed, they were analyzed by nonparametric methods using the Wilcoxon rank sum test. The results from receiver operating characteristic (ROC) curves and Youden analysis were calculated and used to determine the sensitivity and specificity of IGF1, IGF2 and IGFBP2 at discriminating PAH from healthy controls. IGF1, IGF2 and IGFBP2 concentrations were converted to molar concentrations to determine total IGF (IGF1+IGF2) to IGFBP2 ratios as a measure of free IGF (7,12-13). As the ELISA data was not normally distributed, differences in categorical variables were assessed using Wilcoxon signed-rank test, or for continuous variables, Spearman's rank correlation. All continuous variables were reported as median (interquartile range [KM]) in the case of non-normality. Clinical variables and hemodynamic data were correlated with biomarkers using Spearman's rank correlation. Variables were analyzed against hemodynamic data collected within 12 months of collection of the biomarker (N=45) and within 6 months of collection of the biomarker (N=29). Kaplan-Meier methodology was used to estimate survival based on the median concentration of each biomarker. Adjusted analysis was performed by logistic regression using clinically relevant measures as the dependent variables including age, gender, and BSA. A P value less than 0.05 was considered statistically significant. Statistical analysis was performed using Stata (Version 15.1; 2018; StataCorp, LLC, College Station, Tex.).

### Results

Subject Demographics. All PAH patients were diagnosed with WHO group 1 PAH, with serum samples collected at a single time point (enrollment). There were 175 PAH and 75 control enrollees available for analysis. The demographic characteristics of PAH patients and controls are given in Table 1. The median age and gender were not significantly different. The PAH group was 47% idiopathic PAH (IPAH), 46% with associated PAH (APAH), 6% Familial/Hereditary PAH (FPAH/HPAH), 1% with pulmonary vein occlusion. The APAH group was predominately congenital heart disease (55%), with no shunt (13%), unrepaired shunt (24%) and with a repaired shunt (18%). The PAH patients with cardiac hemodynamics (28%) had a mPAP of 53 mmHg and mean PVR of 11.8 WU, consistent with moderate to severe pulmonary hypertension. Functionally, 6MWD was available for 21% of enrollees with a mean of 442 meters. 49% of the PAH cohort were treated with a prostacyclin analogue as another measure of disease severity.

Serum IGF proteins in Pediatric PAH. The limits of detection for the IGF1, IGF2, and IGFBP2, assays were 18.2 ng/mL, 56.3 ng/mL, and 4.4 ng/ml, respectively, with inter-plate coefficients of variation of 5% for IGF1, 2.6% for IGF2, and 3.6% for IGFBP2. The results of the serum IGF1, IGF2, IGFBP2, and total IGF/IGFBP2 concentrations are detailed in Table 2 for both the PAH and control (median and IQR) cohorts. As shown, IGFBP2 and IGF1 concentrations were significantly higher in the PAH group compared with controls (respectively, p=0.0004, 95% CI −109, −33 and p=0.001, 95% CI −44.9 to −10.1), while IGF2 was significantly lower in PAH compared to controls (p=0.001, 95% CI 42.2, 186.8). The present inventors also calculated the serum molar ratio of IGF1+IGF2:IGFBP2 known to reflect free IGF activity (Table 2). The ratio of total IGF to IGFBP2 was significantly lower compared to controls (p=0.0003, 95% CI −8.2 to −2).

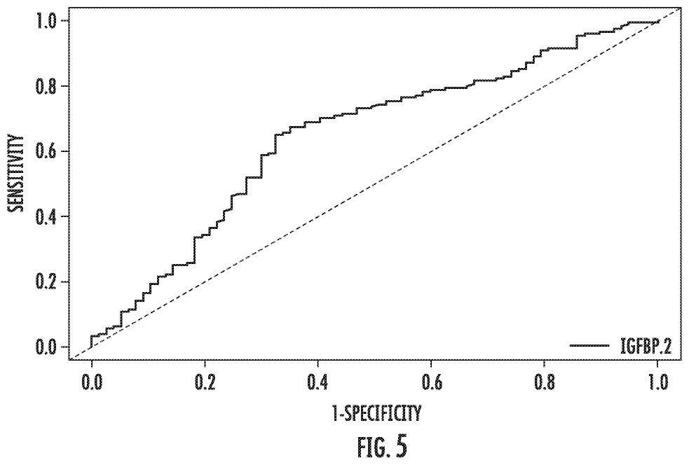

IGFBP2 concentration discriminates PAH from controls. To determine if IGFBP2 could discriminate PAH from controls, the present inventors used IGFBP2 values in the PAH and the control cohorts to generate an ROC curve. Serum IGFBP2 was able to identify the presence of PAH with an AUC of 0.64 (P<0.001), with the mean of sensitivity and specificity maximized at an IGFBP2 threshold of 168 ng/mL (FIG. 5). This IGFBP2 threshold had a sensitivity of 65% and specificity of 57%.

IGF1 and IGFBP2 correlate with hemodynamic markers amongst PAH patients. The present inventors evaluated the correlation of IGF1, IGF2, and IGFBP2 with major hemodynamic variables. Analysis was performed on patients who had a cardiac catheterization within 6 months (N=29, Table 3) of enrollment. IGF1 was significantly negatively correlated with pulmonary vascular resistance (r=−0.549, p=0.003). IGF1 was also negatively correlated with PVRi (r=−0.534, p=0.005). IGFBP2 had a significant positive correlation with PVRi (r=0.669, p<0.001). The ratio of total IGF/IGFBP2 was significantly negatively correlated with PVRi (r=−0.605, p=0.001) respectively. Cardiac output was significantly correlated with IGF1 (r=0.372, p=0.047), and IGFBP2 (r=−0.481, p−0.008); these are in a reciprocal relationship, positive correlation of CO with IGF1, and a negative correlation of CO with IGFBP2.

IGFBP2 concentration correlates with prostacyclin use. As prostacyclin or prostacyclin analogue therapy reflects severe disease, the present inventors explored the relationship of serum IGFBP2 levels and prostacyclin or prostacyclin analogue therapy. In the present PAH cohort, 49% were treated with prostacyclin or a prostacyclin analogue with 35% receiving this treatment by an intravenous or subcutaneous route. Serum IGFBP2 median concentration was significantly higher in the prostacyclin/prostacyclin analogue group (Table 4) and with subcutaneous and intravenous administration compared to other therapies (288.7 ng/mL, IQR 202.3-451.4, p=0.001; 358 ng/mL, 232.6-503, P<0.0001) respectively.

Serum biomarker concentrations and ratios correlate with functional outcomes and mortality. As shown in Table 5 using a linear regression model adjusted for age and gender, higher levels of IGFBP2 were significantly associated with greater risk of mortality. The median IGFBP2 concentration in patients who died was 540 ng/mL compared with 262 ng/mL in survivors (p=0.009). For patients with an IGFBP2 concentration in the top quartile (>379 ng/mL), the relative risk of death was 12.4 (p=0.023, 95% CI 1.4 to 107; NNT (harm) 11.6) compared with patients with an IGFBP2 concentration in the bottom 3 quartiles. IGFBP2 was also strongly associated with worse 6-minute walk distance with an adjusted coefficient of −139 (p=0.01) per 1-natural log unit higher IGFBP2. Total IGF/IGFBP2 was positively associated with 6-minute walk distance with an adjusted coefficient of 90 (p=0.032) per 1-natural log unit higher IGFBP2. IGFBP2 was positively associated with mortality with a log-odds of 1.193 (p=0.016). Thus, the odds ratio of death with 1-natural log unit higher IGFBP2 was 3.3. The total IGF/IGFBP2 molar ratio as a measure of free IGF, was negatively associated with mortality with a log-odds of −1.39 (p=0.016) per 1-natural log unit higher ratio. Thus, the odds ratio of death with 1-natural log unit higher IGF/IGFBP2 was −4.01. Survival analysis was conducted using a Cox proportional hazard model, adjusted for age and gender (Table 2). For 1-natural log unit higher IGFBP2 (Table 2A), the hazard ratio was 8.002, (95% CI 2.07, 30.9; p=0.003) and for 1-natural log unit higher total IGF/IGFBP2 (Table 2B), 0.199 (95% CI 0.069, 0.569; p=0.003) indicating that these markers were independently and strongly predictive of outcomes. Cox proportional hazard model was not significant for IGF1 (hazard ratio 0.782 (95% CI 0.269, 2.272, p=0.65). Cardiac hemodynamics and dyspnea were not significant.

### Discussion

Pulmonary arterial hypertension is a severe disease with an extremely high burden of morbidity and mortality. The present inventors sought to find new circulating biomarkers which may also have a mechanistic role in the pathobiology of PAH. Insulin like growth factors are an interesting target because of their essential role in both myocardial function and metabolism, as well as endothelial growth and development. This study shows that IGFBP2 is elevated in pediatric PAH, with a significant association with disease severity including functional outcomes (6MWD), need for increased treatment (prostacyclin/prostacyclin analogue treatment), and death. As a possible mechanism, IGFBP2 was associated with decreased cardiac output, and increased PVR. The correlation of total IGF/IGFBP2 as a measure of free IGF suggests that worsening IGF availability in PAH may be a mechanism of worsening disease.

Insulin like growth factor 1 is a ubiquitous protein expressed as part of the pituitary growth hormone axis (5,14) where growth hormone stimulates production and release of IGF1 from the liver (5). IGF1 function is mediated by binding the cell surface IGFR1 tyrosine kinase coupled receptor, triggering a signaling cascade that results in a positive growth and metabolic signal (5). IGF1 exerts particular effects in the heart as a growth regulator, with upregulation of IGF1 in animal models of ventricular hypertrophy, and increased pressure or volume overload, while IGF1 deficiency results in heart failure and death (5,15). Equally important, IGF1 is also an endothelial growth factor, modulating vascular tone and nitric oxide production, which when abnormal, contribute to pulmonary hypertension (14). While IGF1 is essential for cardiac function and growth, IGF binding proteins have a role in growth and cellular remodeling of the ventricles, and are associated with worse outcomes after cardiovascular events (9). IGF binding proteins, particularly IGFBP2, are also found to be elevated in lung disease, such as pulmonary fibrosis (7).

While IGFBPs may have some IGF independent actions, the majority of IGF1 actions are mediated by the IGF1/IGF1R interaction. However only about 1% of IGF1 or IGF2 are free, with the remaining 99% bound to IGFBPs (16-17). Although there are seven IGFBPs, serum IGF1 is principally complexed to two binding proteins IGFBP3, the most abundant IGFBP in serum, and acid labile protein (ALP) (17-18). The biology of IGFBP diversity is unclear. While individual IGFBPs each appear to have biologic functions in addition to IGF chaperone mediating functions, IGFBP2 has been shown to be mostly inhibitory (18). In large part IGFBPs serve as a negative growth signal. For example, IGFBP2 mouse transgenic overexpression with a ubiquitous CMV promoter resulted in decreased body mass and muscle weight (17-18). Of interest, IGFBP2 overexpression had a greater effect on somatic growth inhibition in females than males (18). Thus the interplay and dysregulation of IGF1 and IGFBP2, may be important in explaining the increased female gender bias in PAH and why the disease is more severe in men. The IGFBP2 promoter contains progesterone response elements that may play a role in the discrepant gender effects of incidence and severity in PAH (19-22).

In this study the present inventors demonstrate that IGFBP2 is elevated in pediatric PAH and significantly associated with disease severity (6MWD, CO, prostacyclin treatment, mortality). The etiology of the elevated IGFBP2 in pulmonary hypertension is currently unknown. But the pattern seen in these patients is clear; in pulmonary hypertension there is an elevated IGFBP2 concentration, and a lower total IGF concentration, with a lower total IGF/IGFBP2 ratio. IGF and IGFBP2 have not been previously described in the pathobiology of pulmonary hypertension, but their interaction has been extensively described in the hallmark processes of this disease, namely abnormal pulmonary vasculature, with abnormal vascular tone, and resultant right ventricular failure (5,14). The consequences of higher IGFBP2 levels appear to be worsening of disease with signs of increased pulmonary vascular resistance and decreased cardiac output. This is notably the converse correlation with IGF1, a positive effector of cardiovascular function. Their relationship, IGF/IGFBP2 ratio, shows the same pattern, suggesting the deficiency of IGF1, and relative increase in IGFBP2 concentration. IGFBP2 and IGF1 not only have hemodynamic consequences, but are significantly associated with functional outcomes, particularly those associated with ventricular function. As IGFBP2 increases, symptoms of pulmonary hypertension worsen; higher IGFBP2 is associated with shorter 6MWD, as is a lower IGF/IGFBP2 ratio. Taken together, the loss of function of IGF with the increased inhibition by IGFBP2 makes the relationship more striking. The present inventors also observed that patients requiring more medications, particularly IV or subcutaneous infusion, consistently had higher IGFBP2 levels. While this study is unable to determine if the IGFBP2 level was increasing or decreasing with treatment, hemodynamic data, particularly IGFBP2 positively correlating with PVRi, suggest the elevated level is due to worse disease, rather than treatment itself. Finally, those with higher IGFBP2, and lower IGF/IGFBP2 ratio have higher risk of death in multiple adjusted models.

This study is the first to explore IGF1, IGF2 and IGFBP2 in pediatric pulmonary hypertension. The study is primarily cross sectional, with patients enrolled at different phases of disease, between diagnosis and treatment and blood samples collected only at enrollment. A longitudinal analysis of IGF and IGFBP2 in pulmonary hypertension to assess response to treatment and to develop a better prognostic model for outcomes is conducted. There were only 5 deaths in this group, limiting the power to assess mortality, however there was still a significant association with mortality.

Given the essential function of IGF and IGFBP in endothelial growth, maturation and cardiomyocyte growth and function, these proteins may play a key role in the pathogenesis of PAH. These growth factors have potential for describing pathogenesis and suggesting possible new therapeutic targets. This is especially significant since medical therapy is largely aimed at vasodilation and no current therapies specifically target endothelial hyperproliferation or ventricular dysfunction that are so characteristic of severe pulmonary hypertension. In particular embodiments, IGFBP2 is a useful biomarker for diagnosis and prognosis of pediatric pulmonary arterial hypertension.

TABLE 1
Demographics

| | PAH Patient | Control | P |
|---|---|---|---|
| | (N = 175) | (N = 75) | Value |
| Age (Median and IQR) | 13 (8-17) | 12 (8-15) | 0.2 |
| Gender (Female) | 59% | 47% | 0.09 |
| Height (cm) | 117 (91.7-150) | | |
| Weight (kg) | 19.85 (12.1-44.5) | | |
| BSA (m²) | 0.74 (0.52-1.26) | | |
| PAH Subtypes | | | |
| IPAH | 47% (N = 83) | | |
| FPAH | 6% (N = 11) | | |
| Pulmonary vein occlusion | 1% (N = 2) | | |
| APAH | | | |
| APAH: Collagen vascular disease | 2% (N = 4) | | |
| APAH: Portal Hypertension | 2% (N = 3) | | |
| APAH: Congenital heart disease with no shunt | 13% (N = 23) | | |
| APAH: Congenital heart disease with an unrepaired shunt | 24% (N = 43) | | |
| APAH: Congenital heart disease with a repaired shunt | 18% (N = 33) | | |
| Functional Measures in PAH Cases | | | |
| Six Minute Walk Distance, median meters (% missing) | 442 m (43%) | | |
| NYHA Functional Class I/II/III/IV (% missing) | 19/54/49/11 (28%) | | |
| Hemodynamics in PAH Cases (N = 49) | Median (P25-P75) | | |
| Cardiac Output (L/min) | 3.5 (2.4 to 4.6) | | |
| Cardiac Index (L/min/m²) | 3.5 (2.7 to 4.3) | | |
| PVR (WU) | 11.8 (8.0 to 18.5) | | |
| PVRi (WU*m²) | 10.7 (5.4 to 21.7) | | |
| mPAP (mmHg) | 53 (41.5 to 61) | | |
| mRAP (mmHg) | 7 (5.3 to 10) | | |
| mPCWP (mmHg) | 9 (7 to 11) | | |
| PAH Patients Medical Therapy (N = 175) | | | |
| Calcium Channel Blocker | 29 | | |
| Phosphodiesterase Inhibitor | 161 | | |
| Endothelin Receptor Antagonist | 114 | | |
| Prostacyclin or Prostacyclin Analog | 86 | | |
| Phosphodiesterase Inhibitor and Endothelin Receptor Antagonist | 111 | | |
| Phosphodiesterase Inhibitor and Prostacyclin/Prostacyclin Analog | 84 | | |
| Phosphodiesterase inhibitor, endothelin receptor antagonist, and | 69 | | |
| prostacyclin/prostacyclin analog | | | |
| Intravenous/Subcutaneous Prostacyclin/Prostacyclin Analog | 59 | | |
| Phosphodiesterase inhibitor, endothelin receptor antagonist, and IV/SubQ | 45 | | |
| prostacyclin/prostacyclin analog | | | |

TABLE 2

| | Serum Biomarker Concentrations (Median, IQR) | | | |
|---|---|---|---|---|
| | PAH | Control | P Value (95% CI) | |
| IGF1 (ng/mL) | 116 (75-181) | 87 (58-237) | 0.001 | (−45 to −10) |
| IGF2 (ng/mL) | 329 (188-554) | 514 (279-686) | 0.001 | (42 to 187) |
| IGFBP2 (ng/mL) | 264 (168-379) | 175 (123-281) | 0.0004 | (−109 to −33) |
| Total IGF (IGF1 + IGF2) | 65 (42-91) | 83 (50-105) | 0.04 | (−22 to −0.5) |
| (nmol/L) | | | | |
| IGF1/IGFBP2 Molar ratio | 1.9 (0.9-4.8) | 2.5 (1.3-4.4) | 0.44 | (−0.79 to 0.31) |
| | | | *NS | |
| IGF2/IGFBP2 Molar ratio | 50 (23-84) | 13 (5.6-23) | <0.0001 | (23 to 42) |
| Total IGF/IGFBP2 Molar Ratio | 9.2 (4.7-16.3) | 16 (7.7-30) | 0.0003 | (−8.2 to −2) |

TABLE 3
Unadjusted Spearman Correlations of IGF1, IGF2, IGFBP2,
Total IGF/IGFBP2 with hemodynamic and
functional variables (29 patients with Right Heart Cath
within 6 months of enrollment)

| | | | | | | | Total | |
|---|---|---|---|---|---|---|---|---|
| | IGF1 | | IGF2 | | IGFBP2 | | IGF/IGFBP2 | |
| | r | p | r | p | r | p | r | p |
| Six Minute Walk | 0.506 | 0.054 | 0.071 | 0.8 | −0.706 | 0.003 | 0.618 | 0.014 |
| Distance | | | | | | | | |
| Heart Rate | −0.104 | 0.613 | 0.188 | 0.357 | 0.337 | 0.092 | −0.249 | 0.221 |
| Cardiac Output | 0.372 | 0.047 | −0.221 | 0.249 | −0.481 | 0.008 | 0.361 | 0.095 |
| Cardiac Index | −0.03 | 0.879 | −0.054 | 0.785 | 0.061 | 0.759 | −0.188 | 0.339 |
| PVR | −0.549 | 0.003 | 0.107 | 0.595 | 0.611 | 0.001 | −0.521 | 0.005 |
| PVRi | −0.534 | 0.005 | 0.023 | 0.913 | 0.669 | <0.001 | −0.605 | 0.001 |
| mPAP | −0.4 | 0.031 | −0.064 | 0.74 | 0.337 | 0.074 | −0.331 | 0.08 |
| mRAP | −0.312 | 0.099 | −0.13 | 0.501 | 0.247 | 0.197 | −0.21 | 0.275 |
| mPCWP | −0.239 | 0.22 | −0.411 | 0.03 | 0.014 | 0.943 | −0.261 | 0.18 |

TABLE 4
Biomarker: IGFBP2

| | | | | |
|---|---|---|---|---|
| | Median | P25 | P75 | p-value |
| N | (ng/mL) | (ng/mL) | (ng/mL) | (rank Sum) |

Pulmonary Hypertension

| | | | | | |
|---|---|---|---|---|---|
| PH Patient | 176 | 264.1 | 168.4 | 379.1 | P = 0.0004 |
| Healthy Control | 75 | 175.1 | 123.3 | 281.2 | |

Cases Only
On a PDE5 Inhibitor

| | | | | | |
|---|---|---|---|---|---|
| Yes | 165 | 266.5 | 187.7 | 405 | P = 0.0008 |
| No | 14 | 149.8 | 91.03 | 197.1 | |

On an Endothelin Receptor Antagonist

| | | | | | |
|---|---|---|---|---|---|
| Yes | 118 | 269.3 | 186.3 | 417.5 | P = 0.0248 |
| No | 61 | 244.7 | 138.1 | 343.6 | |

On a Prostacyclin Analog

| | | | | | |
|---|---|---|---|---|---|
| Yes | 88 | 288.7 | 202.3 | 451.4 | P = 0.0011 |
| No | 91 | 246.7 | 132 | 319.3 | |

On an IV or SubQ Prostacyclin Analog

| | | | | | |
|---|---|---|---|---|---|
| Yes | 61 | 358 | 232.6 | 503 | P < 0.0001 |
| No | 118 | 243.4 | 135.4 | 319 | |

On a PDE5 Inhibitor and a Prostacyclin Analog

| | | | | | |
|---|---|---|---|---|---|
| Yes | 84 | 295 | 214.1 | 470.7 | P = 0.0002 |
| No | 96 | 243.4 | 132.7 | 319.2 | |

On a PDE5 Inhibitor, an Endothelin Receptor
Antagonist and a Prostacyclin Analog

| | | | | | |
|---|---|---|---|---|---|
| Yes | 69 | 296.6 | 225.4 | 492.3 | P = 0.0007 |
| No | 111 | 247.1 | 137.5 | 342.8 | |

On a PDE5 Inhibitor, an Endothelin Receptor
Antagonist and an IV or SubQ Prostacyclin Analog

| | | | | | |
|---|---|---|---|---|---|
| Yes | 45 | 367 | 260.7 | 523.3 | P < 0.0001 |
| No | 135 | 246.7 | 149.1 | 342.8 | |

TABLE 5

| IGFBP2 | | IGF1 | | IGF2 | | Total IGF/IGFBP2 | |
|---|---|---|---|---|---|---|---|
| Coefficient | P- | Coefficient | P- | Coefficient | P- | Coefficient | P- |
| (95% CI) | Value | (95% CI) | Value | (95% CI) | Value | (95% CI) | Value |

Adjusted Linear Regression of IGFBP2, IGF1, and Total
IGF/IGFBP2 against functional outcomes (N = 42, patients with RHC
within 1 year of enrollment)

| | | | | | | | | |
|---|---|---|---|---|---|---|---|---|
| Six | −139.14 | 0.01 | 5.26 | 0.93 | 76.4 | 0.24 | 90.2 | 0.032 |
| Minute | (−240, −37) | | (−119, 129) | | (−55.7, 208.6) | | (8.48, 171.9) | |
| Walk | | | | | | | | |
| Distance | | | | | | | | |
| Heart | 6.48 | 0.14 | −0.27 | 0.96 | −2.92 | 0.63 | −5.2 | 0.15 |
| Rate | (−2.26, 15.22) | | (−11.2, 10.7) | | (−14.9, .1) | | (−12.3, 1.9) | |
| Cardiac | 0.23 | 0.49 | −0.04 | 0.92 | −0.41 | 0.34 | −0.25 | 0.37 |
| Output | (−0.45, 0.91) | | (−0.85, 0.77) | | (−1.3, .46) | | (−0.8, 0.3) | |

Adjusted Logistic Regression of IGFBP2, IGF1, and Total IGF/IGFBP2 against functional
outcomes (N = 175, all enrollees)

| | | | | | | | | |
|---|---|---|---|---|---|---|---|---|
| Dyspnea | 0.31 | 0.39 | 0.62 | 0.14 | −0.31 | 0.36 | −0.22 | 0.43 |
| | (−0.39, 1.0) | | (−0.19, 1.43) | | (−0.99, .37) | | (−0.75, 0.32) | |
| Mortality | 1.19 | 0.016 | −1.0 | 0.116 | −0.41 | 0.54 | −1.39 | 0.016 |
| | (0.36, 3.5) | | (0.064, 1.19) | | (−1.7 ,.9) | | (−2.54, -0.26) | |

TABLE 6

| IGFBP2 | | IGF1 | | IGF2 | | Total IGF/IGFBP2 | |
|---|---|---|---|---|---|---|---|
| Coefficient | P- | Coefficient | P- | Coefficient | P- | Coefficient | P- |
| (95% CI) | Value | (95% CI) | Value | (95% CI) | Value | (95% CI) | Value |

Adjusted Linear Regression of IGFBP2, IGF1, and Total IGF/IGFBP2 against hemodynamic
and functional outcomes (N = 42, patients with RHC within 1 year of enrollment)

| | | | | | | | | |
|---|---|---|---|---|---|---|---|---|
| Six | −139.14 | 0.01 | 5.26 | 0.93 | 76.4 | 0.24 | 90.17 | 0.032 |
| Minute | (−240, −37) | | (−119, 129) | | (−55.7, 208.6) | | (8.48, 171.9) | |
| Walk | | | | | | | | |
| Distance | | | | | | | | |
| Heart | 6.48 (−2.3, | 0.14 | −0.27 | 0.96 | −2.92 | 0.63 | −5.18 | 0.15 |
| Rate | 15.2) | | (−11.2, 10.7) | | (−14.9, .1 | | (−12.3, 1.9) | |
| Cardiac | 0.23 (−0.45, | 0.49 | −0.039 | 0.92 | −0.41 | 0.34 | −0.25 | 0.37 |
| Output | 0.92) | | (−0.85, 0.77) | | (−1.3, .46) | | (−0.8, 0.3) | |
| Cardiac | 0.36 (−0.29, | 0.28 | 0.42 | 0.32 | −0.43 | 0.10 | −0.43 | 0.10 |
| Index | 1.0) | | (−0.42, 1.3) | | (−0.94, .09) | | (−0.94, 0.09) | |
| PVR | 2.025 (−1.85, | 0.296 | −2.527 | 0.27 | −2.0 (−5.1, | 0.20 | −2.0 (−5.1, | 0.2 |
| | 5.90) | | (−7.14, 2.1) | | .1) | | 1.1) | |
| PVRi | 3.6 | 0.18 | −1.1 (−8.1, | 0.75 | −4.1 (−8.2, | 0.053 | −4.1 − | 0.05 |
| | (−1.7, 8.8) | | 5.9) | | .06) | | (8.2, 0.06) | |
| mPAP | 4.8 (−2.6, | 0.19 | −5.6 (−14.5, | 0.2 | −4.6 (−10.5, | 0.13 | −4.6 (−10.5, | 0.13 |
| | 12.2) | | 3.2) | | .36) | | 1.4) | |
| mRAP | 0.52 (−1.1, | 0.53 | 0.03 (−1.9, | 0.98 | −0.48 (−1.8, | 0.46 | −0.48 (−1.8, | 0.46 |
| | 2.2) | | 1.9) | | .84) | | 0.8) | |
| mPCWP | 0.43 (−0.87, | 0.51 | −1.3 (−2.8, | 0.076 | −0.75 | 0.14 | −0.75 (−1.8, | 0.14 |
| | 1.7) | | 0.14) | | (−1.77, .27) | | 0.3) | |
| Shunt | −11.3 (−61.9, | 0.65 | 3.7 (−62, | 0.91 | 17.4 (−31, | 0.46 | 17.45 | 0.46 |
| | 39.2 | | 69.6) | | 5.8) | | (−30.9, 65.9) | |
| Stroke | −0.001 | 0.85 | 0.002 | 0.74 | 0.001 | 0.9 | 0.001 | 0.9 |
| Volume | (−0.012, 0.01) | | (−0.012, | | (−0.009, | | (−0.009, 0.01) | |
| | | | 0.016) | | .01) | | | |
| Pulse | 4.78 (−0.04, | 0.052 | −2.5 (−8.5, | 0.39 | −3.5 (−7.4, | 0.08 | −3.45 (−7.4, | 0.08 |
| Pressure | 9.6) | | 3.5) | | .46) | | 0.46) | |
| Pulmonary | −0.26 (−0.6, | 0.11 | 0.17 (−0.24, | 0.4 | 0.19 (−0.07, | 0.14 | 0.19 (−0.07, | 0.14 |
| Arterial | 0.07) | | 0.58) | | .47) | | 0.49) | |
| Compliance | | | | | | | | |
| Transpul- | 4.3 (−3.3, | 0.26 | −4.3 (−13.4, | 0.35 | −3.8 (−9.9, | 0.22 | −3.8 (−9.9, | 0.22 |
| monary | 11.9) | | 4.8) | | .3) | | 2.3) | |
| Gradient | | | | | | | | |
| Diastolic | 2.35 (−4.5, | 0.49 | −3.5 (−11.6, | 0.38 | −2.3 (−7.8, | 0.4 | −2.3 (−7.8, | 0.4 |
| Gradient | 9.2) | | 4.5) | | .2) | | 3.2) | |

Adjusted Logistic Regression of IGFBP2, IGF1, and Total IGF/IGFBP2 against functional outcomes
(N = 175, all enrollees)

| | | | | | | | | |
|---|---|---|---|---|---|---|---|---|
| Dyspnea | 0.31 (−0.39, | 0.39 | 0.62 (−0.19, | 0.14 | −0.31 | 0.36 | −0.22 (−0.75, | 0.43 |
| | 1.0) | | 1.43) | | (−0.99, .37) | | 0.32) | |
| Mortality | 1.19 (0.36, | 0.016 | −1.0 (0.06, | 0.12 | −0.41 (−1.7, | 0.54 | −1.39 (−2.54, | 0.016 |
| | 3.47) | | 1.2) | | .9) | | −0.26) | |

### REFERENCES

1. Frank B S and Ivy D D. Diagnosis, Evaluation and Treatment of Pulmonary Arterial Hypertension in Children. Children (Basel). 2018; 5.
2. Tuder R M, Archer S L, Dorfmuller P, Erzurum S C, Guignabert C, Michelakis E, Rabinovitch M, Schermuly R, Stenmark K R and Morrell N W. Relevant issues in the pathology and pathobiology of pulmonary hypertension. J Am Coll Cardiol. 2013; 62:D4-12.
3. Tuder R M and Voelkel N F. Angiogenesis and Pulmonary Hypertension: A Unique Process in a Unique Disease. Antioxidants and Redox Signaling. 2002; 4:833-843.
4. Archer S L, Fang Y H, Ryan J J and Piao L. Metabolism and bioenergetics in the right ventricle and pulmonary vasculature in pulmonary hypertension. Pulm Circ. 2013; 3:144-52.
5. Delafontaine P. Insulin-like growth factor I and its binding proteins in the cardiovascular system. Cardiovasc Res. 1995; 30:825-834.
6. Forbes B E, McCarthy P and Norton R S. Insulin-like growth factor binding proteins: a structural perspective. Front Endocrinol (Lausanne). 2012; 3:38.
7. Guiot J, Bondue B, Henket M, Corhay J L and Louis R. Raised serum levels of IGFBP-1 and IGFBP-2 in idiopathic pulmonary fibrosis. BMC Pulm Med. 2016; 16:86.
8. Rhodes C J, Wharton J, Ghataorhe P, Watson G, Girerd B, Howard L S, Gibbs J S R, Condliffe R, Elliot C A, Kiely D G, Simonneau G, Montani D, Sitbon O, Gall H, Schermuly R T, Ghofrani H A, Lawrie A, Humbert M and Wilkins M R. Plasma proteome analysis in patients with pulmonary arterial hypertension: an observational cohort study. The Lancet Respiratory Medicine. 2017; 5:717-726.
9. Motiwala S R, Szymonifka J, Belcher A, Weiner R B, Baggish A L, Gaggin H K, Bhardwaj A and Januzzi J L, Jr. Measurement of novel biomarkers to predict chronic heart failure outcomes and left ventricular remodeling. J Cardiovasc Transl Res. 2014; 7:250-61.
10. Brankovic M, Akkerhuis K M, Mouthaan H, Brugts J J, Manintveld O C, van Ramshorst J, Germans T, Umans V, Boersma E and Kardys I. Cardiometabolic biomarkers and their temporal patterns predict poor outcome in chronic heart failure (Bio-SHiFT study). J Clin Endocrinol Metab. 2018.
11. Gandhi P U, Gaggin H K, Sheftel A D, Belcher A M, Weiner R B, Baggish A L, Motiwala S R, Liu P P and Januzzi J L, Jr. Prognostic usefulness of insulin-like growth factor-binding protein 7 in heart failure with reduced ejection fraction: a novel biomarker of myocardial diastolic function? Am J Cardiol. 2014; 114:1543-9.
12. Alberti C, Cheyenne D, Mercat I, Josserand E, Armoogum-Boizeau P, Tichet J and Leger J. Serum concentrations of insulin-like growth factor (IGF)-1 and IGF binding protein-3 (IGFBP-3), IGF-1/IGFBP-3 ratio, and markers of bone turnover: reference values for French children and adolescents and z-score comparability with other references. Clin Chem. 2011; 57:1424-35.
13. Chen J W, Hojlund K, Beck-Nielsen H, Sandahl Christiansen J, Orskov H and Frystyk J. Free rather than total circulating insulin-like growth factor-I determines the feedback on growth hormone release in normal subjects. J Clin Endocrinol Metab. 2005; 90:366-71.
14. Delafontaine P, Song Y H and Li Y. Expression, Regulation, and Function of IGF-1, IGF-1R, and IGF-1 Binding Proteins in Blood Vessels. Arteriosclerosis, Thrombosis, and Vascular Biology. 2003; 24:435-444.
15. Wood A W, Schlueter P J and Duan C. Targeted knockdown of insulin-like growth factor binding protein-2 disrupts cardiovascular development in zebrafish embryos. Mol Endocrinol. 2005; 19:1024-34.
16. Collett-Solberg P C, P. Genetics, Chemistry, and Function of the IGF/IGFBP System. Endocrine. 2000; 12:121-136.
17. Silha J V and Murphy L J. Insights from insulin-like growth factor binding protein transgenic mice. Endocrinology. 2002; 143:3711-4.
18. Schneider M R, Lahm H, Wu M, Heflich A and Wolf E. Transgenic mouse models for studying the functions of insulin-like growth factor-binding proteins. The FASEB Journal. 2000; Vol. 14:629-640.
19. Russo V C, Azar W J, Yau S W, Sabin M A and Werther G A. IGFBP-2: The dark horse in metabolism and cancer. Cytokine Growth Factor Rev. 2015; 26:329-46.
20. Kwak I, Song S, Blum J L, Simmen RCM and Simmen F A. Enhancer- and Silencer-Like Sequences That Mediate Insulin-Like Growth Factor-Binding Protein-2 Gene Expression in Uterine Cells of Pregnancy. DNA AND CELL BIOLOGY. 2006; 25:6-18.
21. S.Pons and I.Torres-Aleman. Estradiol Modulates Insulin-Like Growth Factor I Receptors and Binding Proteins in Neurons from the Hypothalamus. Journal of Neuroendocrinology. 1993; 5:267-271.
22. 'mez GPC-G, Chowen J A and Garcia-Segura L M. Estradiol and Progesterone Regulate the Expression of Insulin-like Growth Factor-I Receptor and Insulinlike Growth Factor Binding Protein-2 in the Hypothalamus of Adult Female Rats. Journal of Neurobiology. 2000:269-281.

### Example 3: Role of Other IGFBPs in PAH

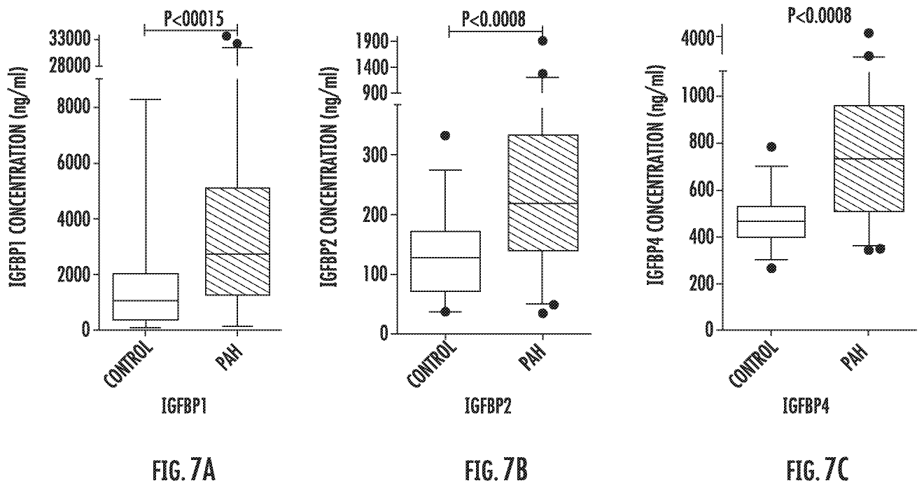

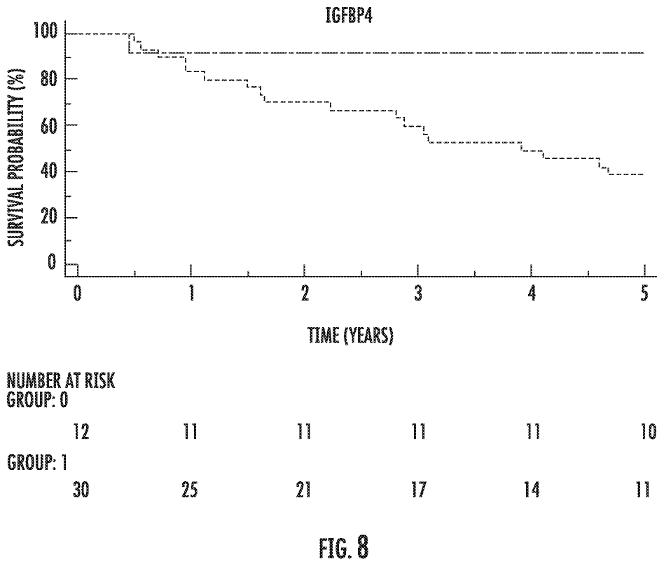

To Explore this, the Present Inventors developed two new multiplex assays for IGFBPs based on their circulating concentrations. To develop the assays, capture antibodies (R&D Systems) for IGFBP1, 4, 5 and IGFBP2, 3, 6 were robotically printed on Meso Scale Discovery (MSD, Gaithersburg, Md.) blank plates for development of electrochemiluminescent ELISA assays. The present inventors pilot tested these assays using JHPH samples (N=42) and non-PH controls (N=38). As shown in FIG. 7, IGFBP1, 2 and 4 were significantly increased in MPH compared to controls. The present inventors also compared the performance of multiplex measurement of IGFBP2 compared to the traditional ELISA and the correlation was 0.98. To determine if these additional IGFBPs have relevance to clinical severity, the present inventors found that IGFBP4 was a significant predictor of survival (FIG. 8) with an unadjusted hazard ratio of 5.8 (CI 2.7-12.6, P=0.002).

In summary, using agnostic mass spectrometry discovery techniques, multiple members of the circulating IGF family of proteins are altered in PAH. The pattern is elevation of specific IGFBPs (at least IGFBP1, 2 and 4). The association of IGF proteins with PAH type, age (pediatric vs adult), response to treatment, clinical worsening, etc. is studied.

### Example 4: Evidence for IGF Proteins as Circulating Pulmonary Hypertension Biomarkers

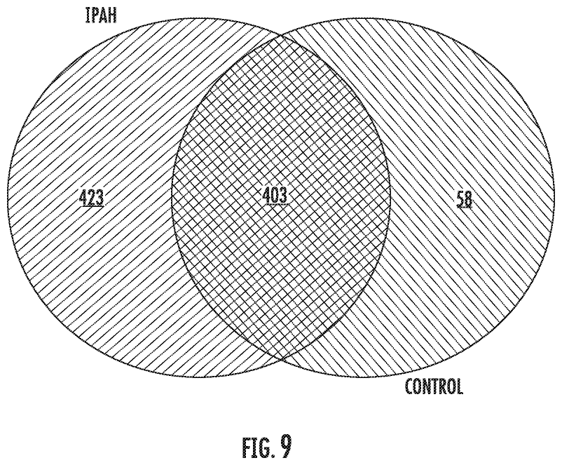

Using mass spectrometry, a total of 826 (FDR 0.047) IPAH and 461 (0.087 FDR) control proteins were identified with 423 proteins unique to the IPAH and 58 unique to control cohorts as shown in the Venn diagram (FIG. 9).

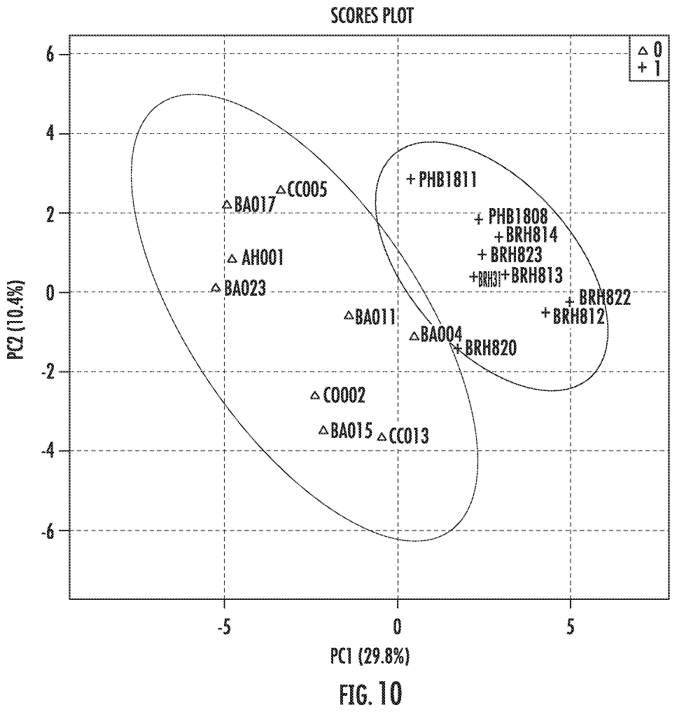

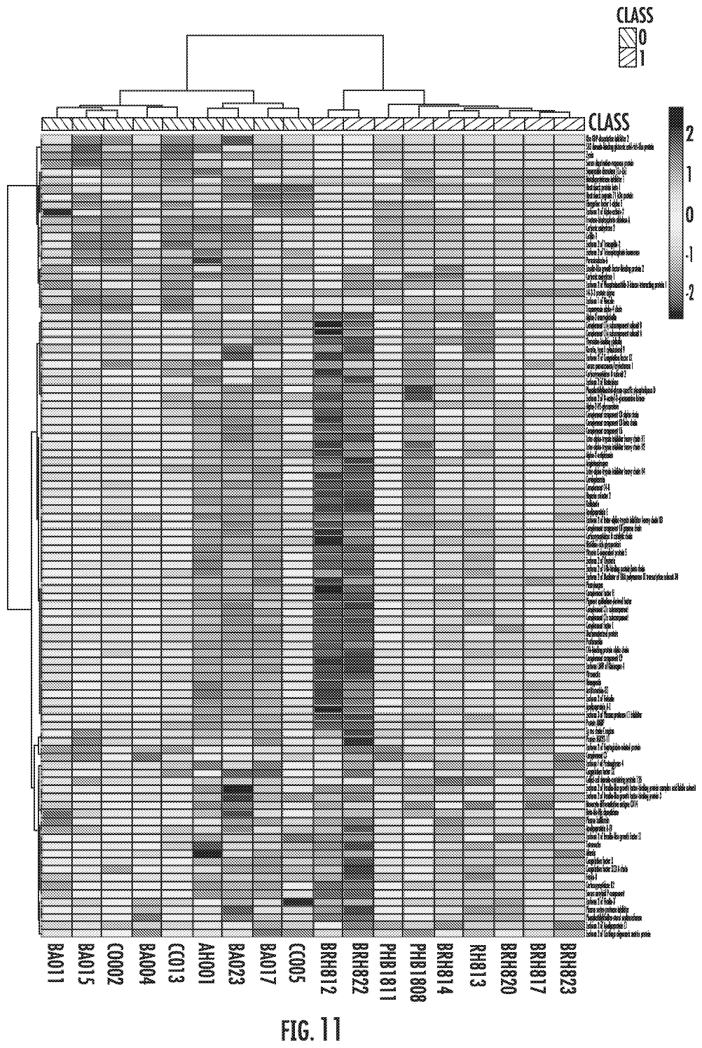

PCA analysis (FIG. 10) and heat maps (FIG. 11) demonstrated that the proteins identified significantly clustered the two cohorts and identified specific protein features discriminating the IPAH and control cohorts.

Using SAM (Statistical Analysis of Microarray), 59 proteins were identified as significant. Insulin-like growth factor-binding protein 2 was in the top ten of most statistically significant candidate proteins.

TABLE 1
Significant protein differences by SAM

| | Gene | | | | |
|---|---|---|---|---|---|
| Protein Name | Name | d. value | stdev | rawp | q. value |
| Carbonic anhydrase 2 | CA2 | −5.3884 | 0.061512 | 0 | 0 |
| Cofilin-1 | CFL1 | −5.1582 | 0.071917 | 0 | 0 |
| Transgelin-2 | TAGLN2 | −4.6342 | 0.073409 | 0 | 0 |
| Elongation factor 1-alpha 1 | EEF1A1 | −4.4127 | 0.077884 | 0 | 0 |
| Fructose-bisphosphate aldolase A | ALDOA | −4.2143 | 0.084278 | 0 | 0 |
| Superoxide dismutase [Cu-Zn] | SOD1 | −3.5273 | 0.087103 | 0.000155 | 0.003313 |
| 14-3-3 protein sigma | SFN | −3.4038 | 0.10419 | 0.000181 | 0.003313 |
| Plasma kallikrein | KLKB1 | 3.2524 | 0.081934 | 0.000311 | 0.003976 |
| Triosephosphate isomerase | TPI1 | −3.1792 | 0.11414 | 0.000311 | 0.003976 |
| Insulin-like growth factor- | IGFBP2 | −3.1713 | 0.091507 | 0.000311 | 0.003976 |
| binding protein 2 | | | | | |
| Serum paraoxonase/arylesterase 1 | PON1 | 3.1316 | 0.080756 | 0.000415 | 0.004098 |
| N-acetylmuramoyl-L-alanine | PGLYRP2 | 3.0811 | 0.098194 | 0.000492 | 0.004098 |
| amidase | | | | | |
| Vinculin | VCL | −3.0662 | 0.11193 | 0.000492 | 0.004098 |
| Carboxypeptidase N subunit 2 | CPN2 | 3.0626 | 0.078304 | 0.000492 | 0.004098 |
| Alpha-actinin-2 | ACYN2 | −3.0454 | 0.08886 | 0.000518 | 0.004098 |
| Carboxypeptidase B2 | CPB2 | 2.9731 | 0.093681 | 0.000596 | 0.004098 |
| Coagulation factor IX | F9 | 2.9707 | 0.087412 | 0.000596 | 0.004098 |
| Complement factor H | CFH | 2.9195 | 0.090907 | 0.000699 | 0.004098 |
| Biotinidase | BTD | 2.8684 | 0.084192 | 0.000751 | 0.004098 |
| Alpha-2-HS-glycoprotein | AHSG | 2.8576 | 0.090928 | 0.000751 | 0.004098 |
| Heparin cofactor 2 | SERPIND1 | 2.8245 | 0.086387 | 0.000803 | 0.004098 |
| Coagulation factor XI | F11 | 2.8224 | 0.097486 | 0.000803 | 0.004098 |
| C4b-binding protein beta chain | C4BPB | 2.7901 | 0.084649 | 0.000855 | 0.004098 |
| Kallistatin | SERPINA4 | 2.7746 | 0.091796 | 0.000855 | 0.004098 |
| Prothrombin | F2 | 2.7602 | 0.092715 | 0.000881 | 0.004098 |
| Corticosteroid-binding globulin | SERPINA6 | 2.7461 | 0.11021 | 0.000984 | 0.004098 |
| Coagulation factor X | F10 | 2.7393 | 0.08893 | 0.00101 | 0.004098 |
| Inter-alpha-trypsin inhibitor | ITIH1 | 2.7234 | 0.091598 | 0.001062 | 0.004098 |
| heavy chain H1 | | | | | |
| Complement component C8 | C8A | 2.7197 | 0.084061 | 0.001088 | 0.004098 |
| alpha chain | | | | | |
| Phosphatidylinositol-glycan- | GPLD1 | 2.7177 | 0.098105 | 0.001088 | 0.004098 |
| specific phospholipase | | | | | |
| Vitamin K-dependent protein S | PROS1 | 2.7021 | 0.090884 | 0.00114 | 0.004098 |
| Serum amyloid P-component | APCS | 2.6894 | 0.095571 | 0.00114 | 0.004098 |
| Gelsolin | GSN | 2.6841 | 0.088156 | 0.001166 | 0.004098 |
| Inter-alpha-trypsin inhibitor | ITIH2 | 2.6814 | 0.094619 | 0.001166 | 0.004098 |
| heavy chain H2 | | | | | |
| Apolipoprotein E | APOE | 2.6752 | 0.10111 | 0.001192 | 0.004098 |
| Histidine-rich glycoprotein | HRG | 2.6608 | 0.092939 | 0.001218 | 0.004098 |
| Inter-alpha-trypsin inhibitor | ITIH3 | 2.6018 | 0.088596 | 0.001554 | 0.00497 |
| heavy chain H3 | | | | | |
| Clusterin | CLU | 2.5801 | 0.087004 | 0.001736 | 0.005414 |
| Inter-alpha-trypsin inhibitor | ITIH4 | 2.5665 | 0.092556 | 0.001814 | 0.005449 |
| heavy chain H4 | | | | | |
| Ceruloplasmin | CP | 2.5605 | 0.096048 | 0.001865 | 0.005449 |
| Complement component C8 beta | C8B | 2.5569 | 0.088992 | 0.001891 | 0.005449 |
| chain | | | | | |
| Complement C1r subcomponent | C1R | 2.5521 | 0.088947 | 0.001917 | 0.005449 |
| Mediator of RNA polymerase II | MED30 | 2.5253 | 0.082581 | 0.002073 | 0.005762 |
| transcription subunit 30 | | | | | |
| C4b-binding protein alpha chain | C4BPA | 2.4873 | 0.098634 | 0.002176 | 0.005798 |
| Complement component C6 | C6 | 2.4669 | 0.082773 | 0.00228 | 0.005847 |
| Carboxypeptidase N catalytic | CPN2 | 2.4544 | 0.090766 | 0.002409 | 0.005926 |
| chain | | | | | |
| Coiled-coil domain-containing | CCDC126 | 2.4459 | 0.12863 | 0.002461 | 0.005939 |
| protein 126 | | | | | |
| Complement C4-B | C4B | 2.42 | 0.1025 | 0.002668 | 0.005941 |
| Monocyte differentiation antigen | CD14 | 2.4176 | 0.10096 | 0.002668 | 0.005941 |
| CD14 | | | | | |
| Apolipoprotein L1 | APOL1 | 2.4137 | 0.12744 | 0.002694 | 0.005941 |
| Complement C1q subcomponent | C1QC | 2.3872 | 0.10352 | 0.002979 | 0.006301 |
| subunit C | | | | | |
| Complement component C8 | C8G | 2.3842 | 0.089255 | 0.003005 | 0.006301 |
| gamma chain | | | | | |
| Complement C1q subcomponent | C1QB | 2.3655 | 0.095748 | 0.003187 | 0.006573 |
| subunit B | | | | | |
| Haptoglobin-related protein | HPR | 2.3582 | 0.11226 | 0.00329 | 0.006627 |
| Isoform F of Proteoglycan 4 | PRG4 | 2.3507 | 0.10701 | 0.003446 | 0.006627 |
| Isoform LMW of Kininogen-1 | KNG1 | 2.3412 | 0.10057 | 0.003523 | 0.006627 |
| Pigment epithelium-derived | SERPINF1 | 2.3369 | 0.087057 | 0.003601 | 0.006627 |
| factor | | | | | |
| Complement component C9 | C9 | 2.3341 | 0.096223 | 0.003627 | 0.006627 |
| Fetuin-B | FETUB | 2.3337 | 0.11095 | 0.003627 | 0.006627 |

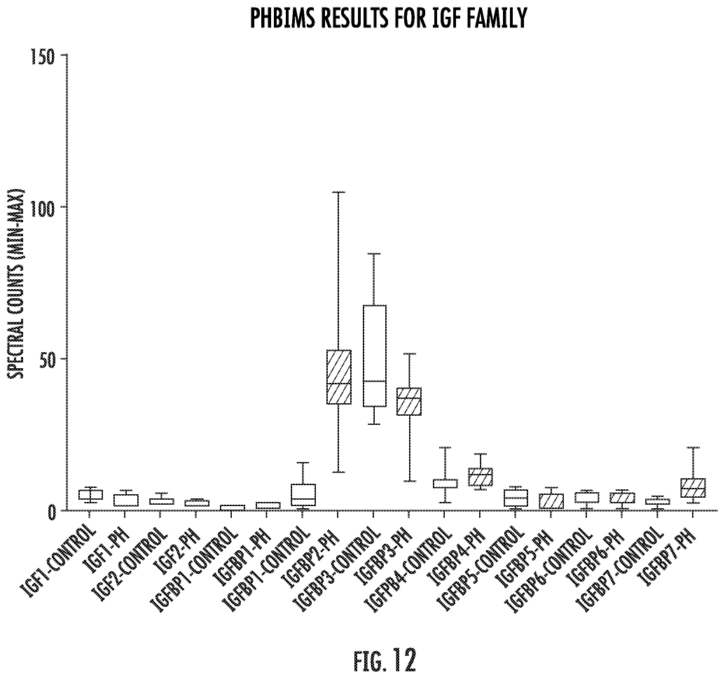

IGFBP2 validation. The present inventors used a different adult PH cohort composed of control and PAH patients from the Johns Hopkins pulmonary hypertension clinic. The present inventors used a commercial ELISA from R & D Systems. As shown IGFBP2 was significantly elevated in adult PAH subjects (N=36) as compared to controls (N=35) as predicted from the present MS results in FIG. 12.

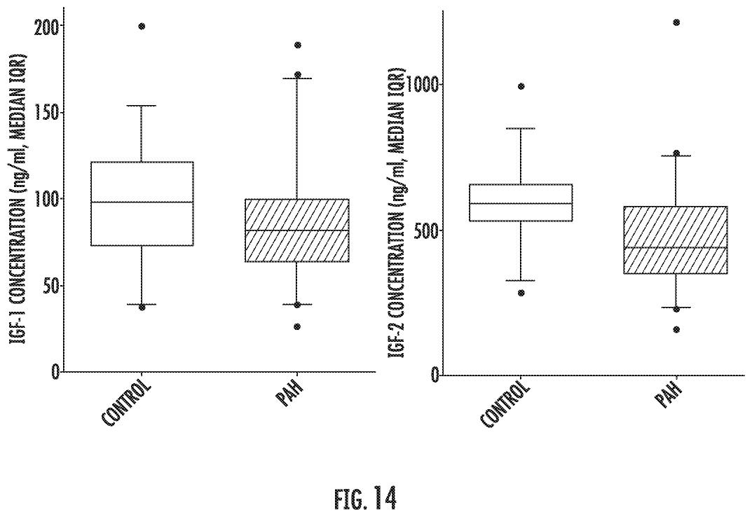

IGF 1 and 2 validation. Our MS data demonstrated that IGF1 and 2 would be decreased in PAH. The present inventors validated these results using the same adult PAH and controls used above. As shown in FIG. 14, both IGF1 and 2 were reduced, but only IGF2 was significantly reduced.

Conclusion: Using a non-biased proteomics discovery approach the present inventors have identified candidate plasma proteins that can distinguish PAH from control and verified elevated IGFBP2 as a new circulating PAH biomarker and in conjunction with the decrease in IGF 1 and 2, could play a significant role in the pathogenesis of PAH.

1. A method comprising the step of measuring insulin-like growth factor binding protein 2 (IGFBP2) in a sample obtained from a subject.

2. The method of claim 1, wherein the sample is a serum sample.

3. The method of claim 1, further comprising measuring insulin-like growth factor 1 (IGF1) and/or IGF2.

4. The method of claim 1, further comprising measuring one or more of IGFBP1, IGFBP3, IGFBP4, IGFBP5, IGFBP6, and IGFBP7.

5. The method of claim 1, further comprising measuring IGFBP1 and IGFBP4.

6. The method of claim 1, further comprising measuring IGF1, IGF2, IGFBP1 and IGFBP4.

7. The method of claim 1, wherein the measuring step is performed using an immunoassay.

8. The method of claim 7, wherein the immunoassay comprises enzyme linked immunosorbent assay (ELISA).

9. The method of claim 1, wherein the subject is suspected of having or has pulmonary arterial hypertension (PAH).

10. A method for identifying subject as having PAH comprising the step of measuring IGFBP2 in a sample obtained from the subject, wherein an increased level of IGFBP2 relative to a control identifies the subject as having PAH.

11. The method of claim 10, wherein the sample is a serum sample.

12. The method of claim 10, further comprising measuring insulin-like growth factor 1 (IGF1) and/or IGF2.

13. The method of claim 1, further comprising measuring one or more of IGFBP1, IGFBP3, IGFBP4, IGFBP5, IGFBP6, and IGFBP7.

14. The method of claim 1, further comprising measuring IGFBP1 and IGFBP4, wherein an increased level of IGFBP1 and IGFBP4 relative to controls identifies the subject as having PAH.

15. The method of claim 1, further comprising measuring IGF1, IGF2, IGFBP1 and IGFBP4 wherein a decreased level of IGF1 and IGF2 and an increased level of IGFBP1 and IGFBP4 relative to controls identifies the subject as having PAH.

16. The method of claim 10, wherein the measuring step is performed using an immunoassay.

17. The method of claim 16, wherein the immunoassay comprises an ELISA.

18. A method comprising the steps of:
(a) detecting an increased level of IGFBP2 relative to a control in a sample obtained from a subject suspected of having PAH; and
(b) treating the subject with a PAH therapy.

19. The method of claim 18, wherein the PAH therapy comprising one or more of endothelial receptor antagonists, prostacyclin pathway agonists, nitric oxide-cyclic guanosine monophosphate (NO-cGMP) enhancers, vasodilators, calcium channel blockers, anticoagulants, oxygen and diuretics.

20. The method of claim 18, wherein the PAH therapy comprises administering prostacyclin or analogs thereof.
